(19) United States
(12) Patent Application Publication
(10) Pub. No.: US 2002/0032238 A1
(43) Pub. Date: Mar. 14, 2002

(54) Biphenylcarboxylic acid amides, the preparation thereof and the use thereof as medicaments

(76) Inventors: Henning Priepke, Warthausen (DE); Norbert Hauel, Schemmerhofen (DE); Leo Thomas, Biberach (DE); Michael Mark, Biberach (DE); Georg Dahmann, Attenweiler (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 09/899,884

(22) Filed: Jul. 6, 2001

Related U.S. Application Data
(60) Provisional application No. 60/220,115, filed on Jul. 24, 2000.

(30) Foreign Application Priority Data
Jul. 8, 2000 (DE) DE 100 33 337.0

(51) Int. Cl. A61K 31/166; C07C 237/48

(52) U.S. Cl. 514/616; 564/155

(57) ABSTRACT

The present invention relates to substituted piperazine derivatives of general formula

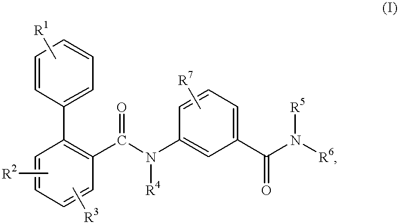

wherein

R¹ to R⁷ are defined herein, the isomers and salts thereof, particularly the physiologically acceptable salts thereof, which are valuable inhibitors of the microsomal triglyceride-transfer protein (MTP), medicaments containing these compounds and their use, as well as the preparation thereof.

### RELATED APPLICATIONS

Benefit of U.S. Provisional Application Ser. No. 60/220,115, filed on Jul. 24, 2000, is hereby claimed and said application is hereby incorporated by reference in its entirety.

### FIELD OF THE INVENTION

The present invention relates to biphenylcarboxylic acid amides of general formula

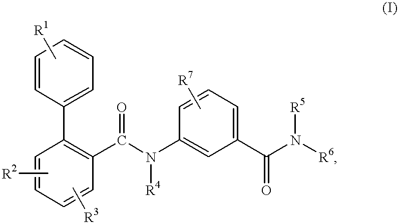

the tautomers, the diastereomers, the enantiomers, the mixtures thereof and the salts thereof, particularly the physiologically acceptable salts thereof which have valuable pharmacological properties, medicaments containing these compounds, their use and processes for preparing them.

The compounds of the above general formula I are valuable inhibitors of the microsomal triglyceride transfer protein (MTP) and are therefore suitable for lowering the plasma level of the atherogenic lipoproteins.

In the above general formula I

R¹, R² and R³, which may be identical or different, in each case denote a hydrogen, fluorine, chlorine or bromine atom, a straight-chain or branched C₁₋₃-alkyl group wherein the hydrogen atoms may be wholly or partially replaced by fluorine atoms, a hydroxy, C₁₋₃-alkoxy, amino, C₁₋₃-alkylamino- or di-(C₁₋₃-alkyl)-amino group,

wherein R¹ and R² in the ortho,ortho′ position of the biphenyl group of formula I together may also denote a carbonyl group,

R⁴ denotes a hydrogen atom or a C₁₋₃-alkyl group,

R⁵ denotes a hydrogen atom or a straight-chain or branched C₁₋₆-alkyl group and

R⁶ denotes a straight-chain or branched C₁₋₆-alkyl group,

an amino, C₁₋₃-alkylamino or di-(C₁₋₃-alkyl)-amino group,

a C₃₋₇-cycloalkylamino or N-(C₁₋₃-alkyl)-C₃₋₇-cycloalkyl-amino group, wherein

in each case the methylene group in the 4 position of a 6- or 7-membered cycloalkyl group may be replaced by an oxygen or sulphur atom or by an imino group optionally substituted by a C₁₋₃-alkyl, phenyl, C₁₋₃-alkyl-carbonyl, benzoyl, phenyl-(C₁₋₃-alkyl)-carbonyl, C₁₋₃-alkyl-aminocarbonyl, di-(C₁₋₃-alkyl)-aminocarbonyl, phenylaminocarbonyl or N-(C₁₋₃-alkyl)-phenylaminocarbonyl group,

an arylamino, N-(C₁₋₃-alkyl)-arylamino, heteroarylamino, N-(C₁₋₃-alkyl)-heteroarylamino, C₁₋₇-alkyl-carbonylamino, N-(C₁₋₃-alkyl)-C₁₋₇-alkyl-carbonylamino, arylcarbonylamino, heteroarylcarbonylamino, N-(C₁₋₃-alkyl)-arylcarbonylamino, N-(C₁₋₃-alkyl)-heteroarylcarbonylamino, C₁₋₈-alkoxy-carbonyl-amino or N-(C₁₋₃-alkyl)-(C₁₋₈-alkoxy)-carbonylamino group,

an aryl, aryl-carbonyl-aryl, aryl-C₁₋₃-alkoxy-aryl or aryl-C₁₋₃-alkyl-aryl group,

a heteroaryl group,

an aryl group substituted by a heteroaryl group,

a C₃₋₇-cycloalkyl or C₃₋₇-cycloalkyl-aryl group, while

in each case the methylene group in the 4 position of a 6- or 7-membered cycloalkyl group may be replaced by an oxygen or sulphur atom or by an imino group optionally substituted by a C₁₋₃-alkyl, phenyl, C₁₋₃-alkyl-carbonyl, benzoyl, phenyl-(C₁₋₃-alkyl)-carbonyl, C₁₋₃-alkyl-aminocarbonyl, di-(C₁₋₃-alkyl)-aminocarbonyl, phenylaminocarbonyl or N-(C₁₋₃-alkyl)-phenylaminocarbonyl group, or

the two hydrogen atoms of the methylene group in the 3 position of a cyclopentyl group or in the 3- or 4-position of a cyclohexyl or cycloheptyl group may be replaced by an n-butylene, n-pentylene, n-hexylene, 1,2-ethylenedioxy or 1,3-propylenedioxy group or

in a 5- or 6-membered cycloalkyl group one or two single bonds separated from each other and from position 1 by at least one bond may each be fused with a phenyl group,

a phenylcarbonylamino-aryl, phenylaminocarbonyl-aryl, N-(C₁₋₃-alkyl)-phenylcarbonylamino-aryl or N-(C₁₋₃-alkyl)-phenylaminocarbonyl-aryl group,

a straight-chain C₁₋₄-alkyl group optionally substituted in the 1 position by a C₃₋₅-cycloalkyl group or a C₁₋₃-alkyl group, which is terminally substituted

by an aryl or heteroaryl group,

by an aryl—C C—, heteroaryl—C C—, aryl—CH═CH— or heteroaryl—CH═CH— group,

by an aryl group which is fused to a heteroaryl group via two adjacent carbon atoms,

by a heteroaryl group which is fused to an aryl or heteroaryl group via two adjacent carbon atoms or, in the case of a 5-membered heteroaryl group, via an imino nitrogen atom and an adjacent carbon atom,

by an aryl group which is substituted

by an aryl or heteroaryl group,

by a C₃₋₇-cycloalkyl group or a 4 to 7 membered cycloalkyleneimino group, which

may each be fused to a phenyl ring via two adjacent carbon atoms or

wherein the two hydrogen atoms of the methylene group in the 3 position of a 5-membered ring or in position 3 or 4 of a 6- or 7-membered ring may be replaced by an n-butylene, n-pentylene, n-hexylene, 1,2-ethylenedioxy or 1,3-propylenedioxy group or by an oxygen atom or

wherein in each case the methylene group in the 4 position of a 6- or 7-membered ring may be replaced by an oxygen or sulphur atom or by an imino group optionally substituted by a C₁₋₃-alkyl, phenyl, C₁₋₈-alkyl-carbonyl, C₁₋₈-alkoxycarbonyl, benzoyl, phenyl-(C₁₋₃-alkyl-carbonyl), C₁₋₃-alkyl-aminocarbonyl, di-(C₁₋₃-alkyl)-aminocarbonyl, phenylaminocarbonyl or N-(C₁₋₃-alkyl)-phenylaminocarbonyl group,

or by a phenylaminosulphonyl or phenylsulphonylamino group,

by a C₃₋₇-cycloalkyl group wherein

in each case the methylene group in the 4 position of a 6- or 7-membered cycloalkyl group may be replaced by an oxygen or sulphur atom or by an imino group optionally substituted

by a C₁₋₃-alkyl, phenyl, C₁₋₈-alkyl-carbonyl, C₁₋₈-alkoxycarbonyl, benzoyl, phenyl-(C₁₋₃-alkylcarbonyl), C₁₋₃-alkylaminocarbonyl, di-(C₁₋₃-alkyl)-aminocarbonyl, phenylaminocarbonyl or N-(C₁₋₃-alkyl)-phenylaminocarbonyl group, by a phenylcarbonylamino-aryl, phenylaminocarbonyl-aryl, N-(C₁₋₃-alkyl)-phenylcarbonylamino-aryl or N-(C₁₋₃-alkyl)-phenylaminocarbonyl-aryl group,

by a heteroarylcarbonylamino-aryl, heteroarylaminocarbonyl-aryl, heteroarylcarbonyl-N-(C₁₋₃-alkyl)-amino-aryl or heteroaryl-N-(C₁₋₃-alkyl)-aminocarbonyl-aryl group,

by a straight-chain or branched C₄₋₇-alkyl-carbonylamino-aryl or N-(C₁₋₃-alkyl)-C₄₋₇-alkyl-carbonylamino-aryl group,

by a C₃₋₇-cycloalkyl-carbonylamino-aryl or N-(C₁₋₃-alkyl)-C₃₋₇-cycloalkyl-carbonylamino-aryl group,

by a C₃₋₇-Cycloalkyl-aminocarbonyl-aryl or N-(C₁₋₃-alkyl)-C₃₋₇-cycloalkyl-aminocarbonyl-aryl group,

by a cycloalkyleneimino-carbonylamino-aryl or cycloalkyleneimino-carbonyl-N-(C₁₋₃-alkyl)-amino-aryl group wherein the cycloalkyleneimino moiety is 4- to 7-membered,

by an aryl-aminocarbonylamino-aryl group wherein one or both amino-hydrogen atoms may each be replaced by a C₁₋₃-alkyl group,

by a hydroxycarbonyl, C₁₋₃-alkoxycarbonyl, C₃₋₇-cyclo-alkyloxycarbonyl, aryloxycarbonyl, heteroaryloxycarbonyl, aryl-C₁₋₃-alkoxycarbonyl or heteroaryl-C₁₋₃-alkoxycarbonyl group or

by an aminocarbonyl, C₁₋₃-alkyl-aminocarbonyl, aryl-C₁₋₃-alkyl-aminocarbonyl, N-(C₁₋₃-alkyl)-aryl-C₁₋₃-alkyl-aminocarbonyl, di-(C₁₋₃-alkyl)-aminocarbonyl, aminocarbonyl-C₁₋₃-alkyl-aminocarbonyl or C₁₋₃-alkoxy-carbonyl-C₁₋₃-alkyl-aminocarbonyl group,

a straight-chain or branched C₂₋₆-alkyl group which is terminally substituted

by a hydroxy, C₁₋₃-alkoxy, aryloxy, heteroaryloxy- aryl-C₁₋₃-alkoxy or heteroaryl-C₁₋₃-alkoxy group,

by an amino, C₁₋₃-alkylamino, di-(C₁₋₃-alkyl)-amino, C₁₋₃-alkyl-carbonylamino, N-(C₁₋₃-alkyl)-C₁₋₃-alkylcarbonylamino, arylcarbonylamino, heteroarylcarbonylamino, N-(C₁₋₃-alkyl)-arylcarbonylamino or N-(C₁₋₃-alkyl)-heteroarylcarbonylamino group,

or R⁵ and R⁶ together with the enclosed nitrogen atom denote a 4- to 7-membered cycloalkyleneimino group wherein the cycloalkylene moiety may be fused to a phenyl ring,

R⁷ denotes a hydrogen, fluorine, chlorine, bromine or iodine atom, a C₁₋₃-alkyl, C₁₋₃-alkoxy, nitro or amino group,

wherein by the term aryl group mentioned above is meant a phenyl, 1-naphthyl or 2-naphthyl group,

by the term heteroaryl group mentioned above is meant a 5-membered heteroaromatic ring linked via a nitrogen or carbon atom, which contains

an imino group, an oxygen or sulphur atom,

an imino group and an oxygen, sulphur or nitrogen atom,

an imino group and two nitrogen atoms or

an oxygen or sulphur atom and two nitrogen atoms,

or a 6-membered heteroaromatic ring linked via a carbon atom which contains one or two nitrogen atoms,

and wherein a 1,4-butadienylene group may be attached both to the abovementioned 5-membered heteroaromatic rings via two adjacent carbon atoms or via an imino nitrogen atom and an adjacent carbon atom and also to the 6-membered heteroaromatic rings in each case via two adjacent carbon atoms and the bicyclic heteroaromatic rings thus formed may also be bonded via a carbon atom of the 1,4- butadienylene group,

a hydrogen atom bonded to a nitrogen atom of the abovementioned 5-membered monocyclic or fused heteroaryl groups may be replaced by a C₁₋₃-alkyl, phenyl, phenyl-C₁₋₃-alkyl, C₁₋₃-alkylcarbonyl, phenylcarbonyl or phenyl-C₁₋₃-alkylcarbonyl group,

all the abovementioned phenyl, aryl and heteroaryl groups as well as aromatic or heteroaromatic parts of molecules in the carbon skeleton may be monosubstituted by a fluorine, chlorine or bromine atom, by a straight-chain or branched C₁₋₄-alkyl group, by a C₃₋₇-cycloalkyl or a 4- to 7-membered cycloalkyleneimino group, while

in each case the methylene group in position 4 of a 6- or 7-membered cycloalkyleneimino group may be replaced by an oxygen or sulphur atom, by a sulphinyl or sulphonyl group or by an imino group optionally substituted by a C₁₋₅-alkyl, phenyl, C₁₋₄-alkyl-carbonyl, C₁₋₄-alkoxy-carbonyl, C₁₋₃-alkyl-aminocarbonyl or di-(C₁₋₃-alkyl)-aminocarbonyl group,

by a trifluoromethyl, phenyl, hydroxy, C₁₋₃-alkoxy, phenyl-C₁₋₃-alkoxy, difluoromethoxy, trifluoromethoxy, amino, C₁₋₃-alkylamino, di-(C₁₋₃-alkyl)-amino, amino-C₁₋₃-alkyl, tert.butoxycarbonylamino-C₁₋₃-alkyl, C₁₋₃-alkylamino-C₁₋₃-alkyl, di-(C₁₋₃-alkyl)-amino-C₁₋₃-alkyl, amino-C₁₋₃-alkyl-carbonyl-amino, C₁₋₃-alkylamino-C₁₋₃-alkyl-carbonyl-amino, di-(C₁₋₃-alkyl)-amino-C₁₋₃-alkyl-carbonyl-amino, phenylamino, N-(C₁₋₃-alkyl)-phenylamino, acetylamino, propionylamino, benzoylamino, N-(C₁₋₃-alkyl)-benzoylamino, acetyl, propionyl, benzoyl, hydroxycarbonyl, C₁₋₄-alkoxycarbonyl, aminocarbonyl, C₁₋₃-alkylamino-carbonyl, 2,2,2-trifluoroethyl-ami-nocarbonyl or di-(C₁₋₃-alkyl)aminocarbonyl group, by a 4- to 7-membered cycloalkyleneimino-carbonyl group or a cyano group or, with the exception of 5-membered heteroaryl groups or heteroaromatic parts of molecules containing more than two heteroatoms, may also be disubstituted by one of the abovementioned substituents and one substituent selected from among fluorine, chlorine, bromine, C₁₋₃-alkyl, trifluoromethyl, C₁₋₃-alkoxy, hydroxy and amino, wherein two adjacent hydrogen atoms in a phenyl group or a phenyl moiety contained in the groups defined above may also be replaced by a methylenedioxy or 1,2-ethylenedioxy group, or may also be trisubstituted by three substituents selected from among fluorine, chlorine and bromine atoms and C₁₋₃-alkyl groups, wherein the substituents may be identical or different and the abovementioned phenyl groups or phenyl moieties may in turn be substituted by a fluorine, chlorine or bromine atom, by a methyl, trifluoromethyl or methoxy group,

in all the abovementioned 4- to 7-membered cycloalkyleneimino groups the cycloalkylene moiety may be fused to a phenyl ring or

one or two hydrogen atoms in each case may be replaced by a C₁₋₃-alkyl group and/or

in each case the methylene group in position 4 of a 6- or 7-membered cycloalkyleneimino group may be substituted by a hydroxycarbonyl, C₁₋₆-alkoxycarbonyl, aminocarbonyl, C₁₋₃-alkylamino-carbonyl, di-(C₁₋₃-alkyl)-aminocarbonyl, phenyl-C₁₋₃-alkylamino or N-(C₁₋₃-alkyl)-phenyl-C₁₋₃-alkylamino group or

may be replaced by an oxygen or sulphur atom, by a sulphinyl or sulphonyl group or by an imino group optionally substituted by a C₁₋₃-alkyl, phenyl, C₁₋₃-alkyl-carbonyl, benzoyl,

phenyl-C₁₋₃-alkyl-carbonyl, C₁₋₃-alkyl-aminocarbonyl, di-(C₁₋₃-alkyl)-aminocarbonyl, phenylaminocarbonyl or N-(C₁₋₃-alkyl)-phenylaminocarbonyl group,

the hydrogen atoms in the C₁₋₃-alkyl and alkoxy groups mentioned in the definition of the above groups may be wholly or partially replaced by fluorine atoms,

additionally any carboxy, amino or imino group present in the abovementioned groups may be substituted by a group which can be cleaved in vivo, and may thus occur in the form of a prodrug group,

and by a group which can be cleaved in vivo from an imino or amino group is meant, for example, a hydroxy group, an acyl group such as the benzoyl or pyridinoyl group or a C₁₋₁₆-alkanoyl group such as the formyl, acetyl, propionyl, butanoyl, pentanoyl or hexanoyl group, an allyloxycarbonyl group, a C₁₋₁₆-alkoxycarbonyl group such as the methoxy-carbonyl, ethoxycarbonyl, propoxycarbonyl, isopropoxycarbonyl, butoxycarbonyl, tert.butoxycarbonyl, pentoxycarbonyl, hexyloxycarbonyl, octyloxycarbonyl, nonyloxycarbonyl, decyloxycarbonyl, undecyloxycarbonyl, dodecyloxycarbonyl or hexadecyloxycarbonyl group, a phenyl-C₁₋₆-alkoxycarbonyl group such as the benzyloxycarbonyl, phenylethoxycarbonyl or phenylpropoxycarbonyl group, a C₁₋₃-alkylsulphonyl-C₂₋₄-alkoxycarbonyl, C₁₋₃-alkoxy-C₂₋₄-alkoxy-C₂₋₄-alkoxycarbonyl or RₑCO—O—(R_{f}CR_{g})—O—CO group wherein

Rₑ denotes a C₁₋₈-alkyl, C₅₋₇-cycloalkyl, phenyl or phenyl- C₁₋₃-alkyl group,

R_{f} denotes a hydrogen atom, a C₁₋₃-alkyl, C₅₋₇-cycloalkyl or phenyl group and

R_{g} denotes a hydrogen atom, a C₁₋₃-alkyl or RₑCO—O—(R_{f}CR_{g})—O group wherein Rₑ to R_{g} are as hereinbefore defined,

whereby the abovementioned ester groups may also be used as a group which can be converted in vivo into a carboxy group.

Moreover, the saturated alkyl and alkoxy moieties which contain more than 2 carbon atoms mentioned hereinbefore and hereinafter in the definitions also include the branched isomers thereof such as, for example, the isopropyl, tert.butyl, isobutyl group, etc., unless otherwise stated.

Preferred compounds of the above general formula I are those wherein

R₁ denotes a hydrogen, fluorine, chlorine or bromine atom or a C₁₋₃-alkyl group wherein the hydrogen atoms may be wholly or partially replaced by fluorine atoms,

R² denotes a hydrogen atom or a C₁₋₃-alkyl group or

R¹ and R² in the ortho, ortho′ position of the biphenyl group of formula I together denote a carbonyl group,

R³, R⁴ and R⁵ which may be identical or different, each denote a hydrogen atom or a C₁₋₃-alkyl group,

R⁶ denotes a straight-chain or branched C₁₋₄-alkyl group,

an amino, C₁₋₃-alkylamino or di-(C₁₋₃-alkyl)-amino group,

a C₃₋₇-cycloalkylamino or N-(C₁₋₃-alkyl)-C₃₋₇-cycloalkyl-amino group, wherein

in each case the methylene group in the 4 position of the cyclohexyl group may be replaced by an oxygen or sulphur atom or by an imino group optionally substituted by a C₁₋₃-alkyl, phenyl, C₁₋₃-alkyl-carbonyl, C₁₋₈-alkoxy-carbonyl, benzoyl, C₁₋₃-alkyl-aminocarbonyl, di-(C₁₋₃-alkyl)-aminocarbonyl, phenyl-aminocarbonyl or N-(C₁₋₃-alkyl)-phenylaminocarbonyl group,

a phenylamino, 1-naphthylamino or 2-naphthylamino group optionally substituted at the nitrogen atom by a C₁₋₃-alkyl group,

a C₁₋₄-alkyl-carbonylamino, phenylcarbonylamino or C₁₋₈-alkoxy-carbonylamino group,

a phenyl, biphenyl, 1-naphthyl, 2-naphthyl, phenylcarbonyl-phenyl, phenyl-C₁₋₃-alkoxyphenyl or phenyl-C₁₋₃-alkylphenyl group which may be substituted in the aromatic moieties in each case by a fluorine, chlorine, bromine or iodine atom, by a straight-chain or branched C₁₋₄-alkyl group, by a trifluoromethyl, hydroxy, C₁₋₃-alkoxy, amino, C₁₋₃-alkylamino, di-(C₁₋₃-alkyl)-amino, acetylamino, benzoylamino, acetyl, benzoyl, C₁₋₃-alkylamino-carbonyl or cyano group,

a heteroaryl group or a heteroaryl-phenyl group,

a C₃₋₇-cycloalkyl or C₃₋₇-cycloalkyl-phenyl group, wherein

in each case the methylene group in the 4 position of the cyclohexyl group may be replaced by an oxygen or sulphur atom or by an imino group optionally substituted by a C₁₋₃-alkyl, phenyl, C₁₋₃-alkylcarbonyl, benzoyl, C₁₋₃-alkyl-aminocarbonyl, di-(C₁₋₃-alkyl)-aminocarbonyl, phenylaminocarbonyl or N-(C₁₋₃-alkyl)-phenylaminocarbonyl group, or

the two hydrogen atoms of the methylene group in the 3 position of a cyclopentyl group or in the 4-position of a cyclohexyl group may be replaced by an n-butylene, n-pentylene, 1,2-ethylenedioxy or 1,3-propylenedioxy group or

in a cyclopentyl or cyclohexyl group one or two single bonds separated from each other and from position 1 by at least one bond may each be fused to a phenyl group,

a phenylcarbonylamino-phenyl, phenylaminocarbonyl-phenyl, N-(C₁₋₃-alkyl)-phenylcarbonylamino-phenyl or N-(C₁₋₃-alkyl)-phenylaminocarbonyl-phenyl group,

a straight-chain C₁₋₄-alkyl group optionally substituted in the 1 position by a cyclopropyl group or a C₁₋₃-alkyl group, which is terminally substituted

by a phenyl, biphenyl, 1-naphthyl or 2-naphthyl group optionally substituted by a fluorine, chlorine, bromine or iodine atom, a straight-chain or branched C₁₋₄-alkyl group, a trifluoromethyl, hydroxy, C₁₋₃-alkoxy, difluoromethoxy, benzyloxy, aminomethyl, amino, C₁₋₃-alkylamino, di-(C₁₋₃-alkyl)-amino, phenylamino, N-(C₁₋₃-alkyl)-phenylamino, acetylamino, acetyl, propionyl, benzoyl, hydroxycarbonyl, C₁₋₄-alkoxycarbonyl, aminocarbonyl, C₁₋₃-alkylamino-carbonyl, di-(C₁₋₃-alkyl)aminocarbonyl, 2,2,2-trifluoroethylaminocarbonyl, pyrrolidinocarbonyl, piperidinocarbonyl or cyano group wherein two adjacent hydrogen atoms may also be replaced by a methylenedioxy or 1,2-ethylenedioxy group,

by a heteroaryl group optionally substituted in the carbon skeleton by a fluorine, chlorine, bromine or iodine atom, by a straight-chain or branched C₁₋₄-alkyl or C₁₋₃-alkoxy group, by a trifluoromethyl, phenyl or cyano group,

by a phenyl—C C— or phenyl—CH═CH— group which may be substituted in the phenyl moiety by a fluorine, chlorine, bromine or iodine atom, by a straight-chain or branched C₁₋₄-alkyl or C₁₋₃-alkoxy group, by a trifluoromethyl, dimethylamino, phenyl or cyano group,

by an indolyl, benzimidazolyl, quinolinyl, isoquinolinyl, quinoxalinyl or quinazolinyl group bonded via a carbon atom or, in the case of the first two groups, via a nitrogen atom, by a phenyl group which is substituted by a heteroaryl group optionally substituted in the carbon skeleton by a fluorine, chlorine, bromine or iodine atom, by a straight-chain or branched C₁₋₄-alkyl group, by a C₃₋₇-cycloalkyl, trifluoromethyl, phenyl or cyano group,

by a C₅₋₆-cycloalkyl group or a 5- or 6-membered cycloalkyleneimino group which

may be fused to a phenyl ring in each case via two adjacent carbon atoms or

wherein the two hydrogen atoms of the methylene group in the 3 position of a 5-membered ring or in the 4 position of a 6-membered ring may be replaced by an n-butylene, n-pentylene, n-hexylene, 1,2-ethylenedioxy or 1,3-propylenedioxy group or by an oxygen atom or

wherein the methylene group in the 4 position of a 6-membered ring may be replaced by an oxygen or sulphur atom or by an imino group optionally substituted by a C₁₋₃-alkyl, phenyl, C₁₋₄-alkyl-carbonyl, C₁₋₄-alkoxy-carbonyl or benzoyl group,

by a phenylaminosulphonylphenyl or phenylsulphonyl-aminophenyl group,

by a C₃₋₇-cycloalkyl group, wherein

in each case the methylene group in the 4 position of the cyclohexyl group may be replaced by an oxygen or sulphur atom or by an imino group optionally substituted by a C₁₋₃-alkyl, phenyl, C₁₋₃-alkyl-carbonyl, benzoyl, C₁₋₃-alkyl-aminocarbonyl, di-(C₁₋₃-alkyl)-aminocarbonyl, phenylaminocarbonyl or N-(C₁₋₃-alkyl)-phenylaminocarbonyl group,

by a phenylcarbonylamino-phenyl, phenylaminocarbonyl-phenyl, N-(C₁₋₃-alkyl)-phenylcarbonylamino-phenyl or N-(C₁₋₃-alkyl)-phenylaminocarbonyl-phenyl group, phenyl-C₁₋₃-alkyl-aminocarbonyl-phenyl, N-(C₁₋₃-alkyl)-phenyl-C₁₋₃-alkyl-aminocarbonyl-phenyl,

C₃₋₇-cycloalkyl-carbonylamino-phenyl,

N-(C₁₋₃-alkyl)-C3-7-aycloalkyl-carbonylamino-phenyl,

C3-7-cycloalkyl-aminocarbonyl-phenyl,

N-(C₁₋₃-alkyl)-C3-7-cycloalkyl-aminocarbonyl-phenyl, C₄₋₆-alkyl-carbonylamino-phenyl,

N-(C₁₋₃-alkyl)-C₄₋₆-alkyl-carbonylamino-phenyl, heteroarylcarbonylamino-phenyl,

N-(C₁₋₃-alkyl)-heteroarylcarbonylamino-phenyl, pyrrolidinocarbonyl-amino-phenyl, piperidinocarbonyl-amino-phenyl, N-(C₁₋₃-alkyl)-pyrrolidinocarbonyl-amino-phenyl,

N-(C₁₋₃-alkyl)-piperidinocarbonyl-amino-phenyl, phenylaminocarbonylamino-phenyl,

N-(C₁₋₃-alkyl)-phenylaminocarbonylamino-phenyl or

N,N-di-(C₁₋₃-alkyl)-phenylaminocarbonylamino-phenyl group,

by a hydroxycarbonyl, C₁₋₃-alkoxycarbonyl, phenyloxycarbonyl or heteroaryloxycarbonyl group,

by an aminocarbonyl, C₁₋₃-alkyl-aminocarbonyl, benzyl-aminocarbonyl, di-(C₁₋₃-alkyl)-aminocarbonyl, aminocarbonyl-C₁₋₃-alkyl-aminocarbonyl or C₁₋₃-alkoxy-carbonyl-C₁₋₃-alkyl-aminocarbonyl group,

a straight-chain C2-3-alkyl group which is terminally substituted

by a hydroxy, C₁₋₃-alkoxy, phenoxy or phenyl-C₁₋₃-alkoxy group or

by an amino, C₁₋₃-alkylamino, di-(C₁₋₃-alkyl)-amino, C₁₋₃-alkyl-carbonylamino, N-(C₁₋₃-alkyl)-C₁₋₃-alkyl-carbonylamino, phenylcarbonylamino or N-(C₁₋₃-alkyl)phenylcarbonylamino group,

or R⁵ and R⁶ together with the enclosed nitrogen atom denote a pyrrolidino or piperidino group which

may each be fused via two adjacent carbon atoms to a phenyl ring optionally substituted by one or two C₁₋₃-alkoxy groups, by an amino, C₁₋₃-alkylamino, acetylamino, aminomethylcarbonylamino or dimethylaminomethylcarbonylamino group,

or a piperazino, morpholino or thiomorpholino group, while the nitrogen atom in the 4 position of the piperazino group may be substituted by a C₁₋₃-alkyl, phenyl, C₁₋₃-alkylcarbonyl, benzoyl, C₁₋₃-alkyl-aminocarbonyl or phenylaminocarbonyl group, and

R⁷ denotes a hydrogen, fluorine, chlorine or bromine atom or a C₁₋₃-alkyl group or a nitro or amino group,

while, unless otherwise specified, by the term heteroaryl group mentioned above is meant a 2-pyridyl, 3-pyridyl, 4-pyridyl, pyrazinyl, 2-pyrimidinyl, 4-pyrimidinyl, 5-pyrimidinyl, 3-pyridazinyl, 4-pyridazinyl, 1-pyrrolyl, 2-pyrrolyl, 3-pyrrolyl, 2-furyl, 3-furyl, 2-thienyl, 3-thienyl, 1-imidazolyl, 2-imidazolyl, 4-imidazolyl, 1-pyrazolyl, 3-pyrazolyl, 4-pyrazolyl, 2-thiazolyl, 4-thiazolyl, 5-thiazolyl, [1,2,3]-thiadiazol-4-yl, benzimidazol-2-yl, benzimidazol-5-yl, or imidazo-[1,2-a]pyridin-2-yl group optionally substituted in the carbon skeleton by up to three C₁₋₃-alkyl groups and

all the abovementioned phenyl groups, heteroaryl groups, aromatic or heteroaromatic parts of molecules may optionally additionally be substituted in the carbon skeleton by a fluorine, chlorine or bromine atom, by a cyano group or by a straight-chain or branched C₁₋₃-alkyl or trifluoromethyl group,

and/or a hydrogen atom bonded to a nitrogen atom of a heteroaryl group or heteroaromatic part of a molecule may be replaced by a C₁₋₃-alkyl, phenyl or C₁₋₃-alkylcarbonyl group,

the tautomers, the diastereomers, the enantiomers, the mixtures thereof and the salts thereof.

Particularly preferred compounds of the above general formula I are those wherein

R¹ denotes a hydrogen, fluorine, chlorine or bromine atom, a C₁₋₃-alkyl or trifluoromethyl group,

R² denotes a hydrogen atom or a C₁₋₃-alkyl group or

R¹ and R² in the ortho, ortho′ position of the biphenyl group of formula I may together also denote a carbonyl group,

R³ and R⁴ each denote a hydrogen atom,

R⁶ denotes a hydrogen atom or a C₁₋₃-alkyl group,

R⁶ denotes a straight-chain or branched C₁₋₄-alkyl group,

a phenyl, biphenyl or phenyl-C₁₋₃-alkylphenyl group,

a straight-chain C₁₋₃-alkyl group optionally substituted in the 1 position by a cyclopropyl group or a C₁₋₃-alkyl group which is terminally substituted

by a phenyl or biphenyl group which may be substituted in each case by a fluorine, chlorine or bromine atom, by a straight-chain or branched C₁₋₄-alkyl group, by a trifluoromethyl, hydroxy, phenylamino or N-(C₁₋₃-alkyl)-phenylamino group,

by a 2-pyridyl, 3-pyridyl, 4-pyridyl or 1H-benzimidazol-2-yl group,

by a phenyl group which is substituted by a 2-pyridyl, 3-pyridyl, 4-pyridyl, pyrazinyl, 2-pyrimidinyl, 4-pyrimidinyl, 5-pyrimidinyl, 3-pyridazinyl, 4-pyridazinyl, 1-pyrrolyl, 2-pyrrolyl, 3-pyrrolyl, 1-imidazolyl, 2-imidazolyl, 4-imidazolyl, 1-pyrazolyl, 3-pyrazolyl, 4-pyrazolyl, 2-thiazolyl, 4-thiazolyl, 5-thiazolyl, [1,2,3] -thiadiazol-4-yl, benzimidazol-2-yl or imidazo-[1,2-a]pyridin-2-yl group, wherein the abovementioned heteroaromatic groups may be substituted in the carbon skeleton by a fluorine, chlorine or bromine atom, by a phenyl, C₁₋₄-alkyl, trifluoromethyl, C₁₋₃-alkoxy, dimethylamino or C₃₋₇-cycloalkyl group,

by a phenyl group which is substituted by a pyrrolidino or piperidino group optionally fused to a phenyl group,

by a phenyl—C C— group which may be substituted in the phenyl moiety by a fluorine, chlorine or bromine atom, by a straight-chain or branched C₁₋₄-alkyl or C₁₋₃-alkoxy group, by a trifluoromethyl or phenyl group,

by a 4-piperidinyl group optionally substituted at the nitrogen atom by a C₁₋₃-alkyl, C₁₋₃-alkyl-carbonyl, benzoyl, C₁₋₃-alkylaminocarbonyl, di-(C₁₋₃-alkyl)-aminocarbonyl, phenylamino-carbonyl or N-(C₁₋₃-alkyl)-phenylaminocarbonyl group,

by a phenylcarbonylamino-phenyl, phenylaminocarbonyl-phenyl, N-(C₁₋₃-alkyl)-phenylcarbonylamino-phenyl or N-(C₁₋₃-alkyl)-phenylaminocarbonyl-phenyl group optionally substituted in the terminal phenyl moieties by a C₁₋₃-alkyl group or

by a heteroaryl-carbonylamino-phenyl or N-(C₁₋₃-alkyl)-heteroaryl-carbonylamino-phenyl group, wherein the heteroaryl moiety is selected from among 2-pyridyl, 3-pyridyl, 4-pyridyl, pyrazinyl, 2-pyrimidinyl, 4-pyrimidinyl, 5-pyrimidinyl, 3-pyridazinyl, 4-pyridazinyl, 1-pyrrolyl, 2-pyrrolyl, 3-pyrrolyl, 1-imidazolyl, 2-imidazolyl, 4-imidazolyl, 1-pyrazolyl, 3-pyrazolyl, 4-pyrazolyl, 2-thiazolyl, 4-thiazolyl, 5-thiazolyl and [1,2,3]-thiadiazol-4-yl, wherein a hydrogen atom bound to a nitrogen atom of a heteroaromatic group and/or a hydrogen atom bound to a carbon atom of a heteroaromatic group may in each case be replaced by a C₁₋₃-alkyl group, and

R⁷ denotes a hydrogen, fluorine, chlorine or bromine atom, a C₁₋₃-alkyl group or an amino group,

while all the abovementioned phenyl groups, heteroaryl groups, aromatic or heteroaromatic parts of molecules in the carbon skeleton may optionally additionally be substituted by a fluorine, chlorine or bromine atom, by a straight-chain or branched C₁₋₃-alkyl group, by a cyano or a trifluoromethyl group,

the tautomers, the diastereomers, the enantiomers, the mixtures thereof and the salts thereof.

The following are mentioned as examples of particularly preferred compounds:

(a) N-[4-(3-Methyl-5-phenyl-pyrazol-1-yl)-phenylmethyl]-3-(4′-trifluoromethylbiphenyl-2-carbonylamino)-benzoic acid amide

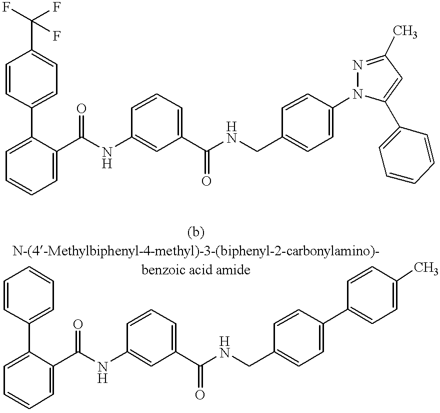

(c) N-[4-(Pyridin-2-yl-carbonylamino)-phenylmethyl]-3-(4′-trifluoromethylbiphenyl-2-carbonylamino)- benzoic acid amide,

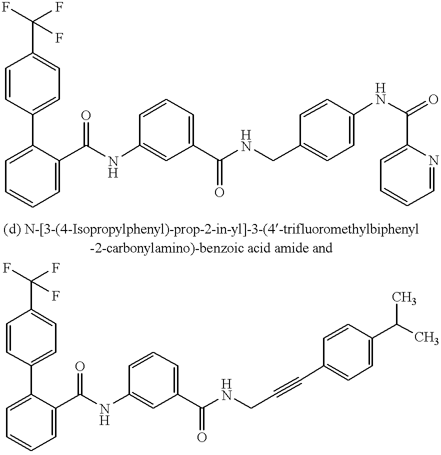

(e) N-[4-(1,2,3 ,4-Tetrahydrochinolin-1-yl)-phenylmethyl]-3-(4′-trifluormethylbiphenyl-2-carbonylamino)-benzoic acid amide

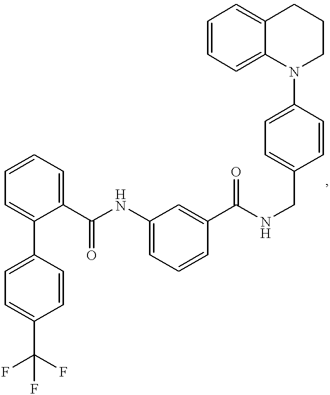

and the salts thereof.

According to the invention, the new compounds are obtained by methods known from the literature, e.g. by the following methods:

a. reacting a compound of general formula

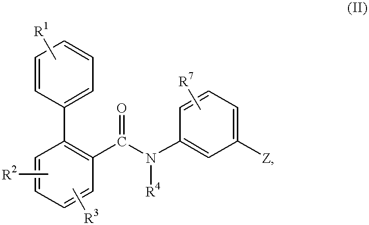

wherein

R¹ to R⁴ and R⁷ are as hereinbefore defined, and Z denotes a carboxy group or a reactive derivative of a carboxy group,

with an amine of general formula

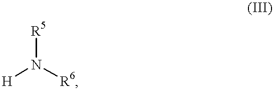

wherein

R⁵ and R⁶ are as hereinbefore defined.

The reaction is expediently carried out with a corresponding halide or anhydride of general formula III in a solvent such as methylene chloride, chloroform, carbon tetrachloride, ether, tetrahydrofuran, dioxane, benzene, toluene, acetonitrile or sulpholane optionally in the presence of an inorganic or organic base at temperatures between −20 and 200° C., but preferably at temperatures between −10 and 160° C. However, it may also be carried out with the free acid, optionally in the presence of an acid-activating agent, e.g. propanephosphonic acid cycloanhydride or 2-(1H-benzotriazol-1-yl)-1,1,3,3-tetramethyluronium-tetrafluoroborate (TBTU), or a dehydrating agent, e.g. in the presence of isobutyl chloroformate, thionyl chloride, trimethylchlorosilane, hydrogen chloride, sulphuric acid, methanesulphonic acid, p-toluenesulphonic acid, phosphorus trichloride, phosphorus pentoxide, N,N′-dicyclohexylcarbodiimide, N,N′-dicyclohexyl carbodiimide/N-hydroxysuccinimide or 1-hydroxy-benzotriazole, N,N′-carbonyldiimidazole or N,N′-thionyldiimidazole or triphenylphosphine/carbon tetrachloride, at temperatures between −20 and 200° C., but preferably at temperatures between −10 and 160° C.

b. reacting a compound of general formula

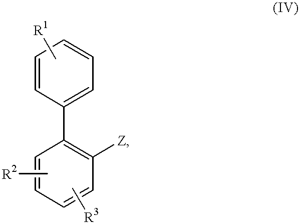

wherein

R¹ to R³ are as hereinbefore defined, and Z denotes a carboxy group or a reactive derivative of a carboxy group,

with an amine of general formula

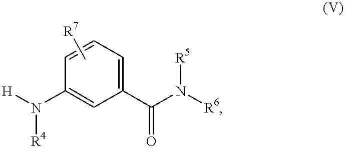

wherein

R⁴ and R⁷ are as hereinbefore defined.

The reaction may be carried out in accordance with the conditions mentioned above for method (a).

If according to the invention a compound of general formula I is obtained which contains an amino, alkylamino or imino group, this may be converted by acylation or sulphonylation into a corresponding acyl or sulphonyl compound of general formula I or

if a compound of general formula I is obtained which contains an amino, alkylamino or imino group, this may be converted by alkylation or reductive alkylation into a corresponding alkyl compound of general formula I or

if a compound of general formula I is obtained which contains a carboxy group, this may be converted by esterification into a corresponding ester of general formula I or

if a compound of general formula I is obtained which contains a carboxy or ester group, this may be converted by amidation into a corresponding amide of general formula I.

The subsequent acylation or sulphonylation is optionally carried out in a solvent or mixture of solvents such as methylene chloride, dimethylformamide, benzene, toluene, chlorobenzene, tetrahydrofuran, benzene/tetrahydrofuran or dioxane with a corresponding acyl or sulphonyl derivative, optionally in the presence of a tertiary organic base or in the presence of an inorganic base or in the presence of a dehydrating agent, e.g. in the presence of isobutyl chloroformate, thionyl chloride, trimethylchlorosilane, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide or 1-hydroxybenzotriazole and optionally also in the presence of 4-dimethylamino-pyridine, N,N′-carbonyldiimidazole or triphenylphosphine/carbon tetrachloride, expediently at temperatures between 0 and 150° C., preferably at temperatures between 0 and 80° C.

The subsequent alkylation is optionally carried out in a solvent or mixture of solvents such as methylene chloride, dimethylformamide, benzene, toluene, chlorobenzene, tetrahydrofuran, benzene/tetrahydrofuran or dioxane with an alkylating agent such as a corresponding halide or sulphonic acid ester, e.g. with methyl iodide, ethyl bromide, dimethylsulphate or benzyl chloride, optionally in the presence of a tertiary organic base or in the presence of an inorganic base, expediently at temperatures between 0 and 150° C., preferably at temperatures between 0 and 100° C.

The subsequent reductive alkylation is carried out with a corresponding carbonyl compound such as formaldehyde, acetaldehyde, propionaldehyde, acetone or butyraldehyde in the presence of a complex metal hydride such as sodium borohydride, lithium borohydride or sodium cyanoborohydride, expediently at a pH of 6-7 and at ambient temperature or in the presence of a hydrogenation catalyst, e.g. with hydrogen in the presence of palladium/charcoal, at a hydrogen pressure of 1 to 5 bar. However, the methylation is preferably carried out in the presence of formic acid as reducing agent at elevated temperatures, e.g. at temperatures between 60 and 120° C.

The subsequent esterification is optionally carried out in a solvent or mixture of solvents such as methylene chloride, dimethylformamide, benzene, toluene, chlorobenzene, tetrahydrofuran, benzene/tetrahydrofuran or dioxane or most advantageously in a corresponding alcohol optionally in the presence of an acid such as hydrochloric acid or in the presence of a dehydrating agent, e.g. in the presence of isobutyl chloroformate, thionyl chloride, trimethylchlorosilane, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide or 1-hydroxy-benztriazole and optionally also in the presence of 4-dimethylamino-pyridine, N,N′-carbonyldiimidazole or triphenylphosphine/carbon tetrachloride, conveniently at temperatures between 0 and 150° C., preferably at temperatures between 0 and 80° C.

The subsequent amidation is carried out by reacting a corresponding reactive carboxylic acid derivative with a corresponding amine, optionally in a solvent or mixture of solvents such as methylene chloride, dimethylformamide, benzene, toluene, chlorobenzene, tetrahydrofuran, benzene/tetrahydrofuran or dioxane, while the amine used may act as solvent at the same time, optionally in the presence of a tertiary organic base or in the presence of an inorganic base or with a corresponding carboxylic acid in the presence of a dehydrating agent, e.g. in the presence of isobutyl chloroformate, thionyl chloride, trimethylchlorosilane, sulphuric acid, methanesulphonic acid, p-toluenesulphonic acid, phosphorus trichloride, phosphorus pentoxide, O-(benzotriazol-1-yl)-N,N,N′,N′-tetramethyluronium-tetrafluoroborate, N,N′-dicyclohexylcarbodiimide, N,N′-dicyclohexylcarbodiimide/N-hydroxysuccinimide or 1-hydroxy-benztriazole and optionally also in the presence of 4-dimethylamino-pyridine, N,N′-carbonyldiimidazole or triphenylphosphine/carbon tetrachloride, conveniently at temperatures between 0 and 150° C., preferably at temperatures between 0 and 80° C.

In the reactions described hereinbefore, any reactive groups present such as hydroxy, carboxy, amino, alkylamino or imino groups may be protected during the reaction by conventional protecting groups which are cleaved again after the reaction.

For example, a protecting group for a hydroxy group may be a trimethylsilyl, tert.butyl-dimethylsilyl, acetyl, benzoyl, methyl, ethyl, tert.butyl, trityl, benzyl or tetrahydropyranyl group,

a protecting group for a carboxyl group may be a trimethylsilyl, methyl, ethyl, tert.butyl, benzyl or tetrahydropyranyl group and

protecting groups for an amino, alkylamino or imino group may be a formyl, acetyl, trifluoroacetyl, ethoxycarbonyl, tert.butoxycarbonyl, benzyloxycarbonyl, benzyl, methoxy-benzyl or 2,4-dimethoxybenzyl group and additionally, for the amino group, a phthalyl group.

Any protecting group used is optionally subsequently cleaved for example by hydrolysis in an aqueous solvent, e.g. in water, isopropanol/water, acetic acid/water, tetrahydrofuran/water or dioxane/water, in the presence of an acid such as trifluoroacetic acid, hydrochloric acid or sulphuric acid or in the presence of an alkali metal base such as sodium hydroxide or potassium hydroxide or aprotically, e.g. in the presence of iodotrimethylsilane, at temperatures between 0 and 120° C., preferably at temperatures between 10 and 100° C. However, a silyl group may also be cleaved using tetrabutylammonium fluoride as described hereinbefore.

However, a benzyl, methoxybenzyl or benzyloxycarbonyl group is cleaved, for example hydrogenolytically, e.g. with hydrogen in the presence of a catalyst such as palladium/charcoal in a suitable solvent such as methanol, ethanol, ethyl acetate or glacial acetic acid, optionally with the addition of an acid such as hydrochloric acid at temperatures between 0 and 100° C., but preferably at temperatures between 20 and 60° C., and at a hydrogen pressure of 1 to 7 bar, but preferably 3 to 5 bar. A 2,4-dimethoxybenzyl group, however, is preferably cleaved in trifluoroacetic acid in the presence of anisole.

A tert.butyl or tert.butyloxycarbonyl group is preferably cleaved by treating with an acid such as trifluoroacetic acid or hydrochloric acid or by treating with iodotrimethylsilane, optionally using a solvent such as methylene chloride, dioxane, methanol or diethylether.

A trifluoroacetyl group is preferably cleaved by treating with an acid such as hydrochloric acid, optionally in the presence of a solvent such as acetic acid at temperatures between 50 and 120° C. or by treating with sodium hydroxide solution, optionally in the presence of a solvent such as tetrahydrofuran at temperatures between 0 and 50° C.

A phthalyl group is preferably cleaved in the presence of hydrazine or a primary amine such as methylamine, ethylamine or n-butylamine in a solvent such as methanol, ethanol, isopropanol, toluene/water or dioxane at temperatures between 20 and 50° C.

Moreover, the compounds of general formula I obtained may be resolved into their enantiomers and/or diastereomers, as mentioned hereinbefore. Thus, for example, cis/trans mixtures may be resolved into their cis and trans isomers, and compounds with at least one optically active carbon atom may be separated into their enantiomers.

Thus, for example, the cis/trans mixtures obtained may be resolved by chromatography into the cis and trans isomers thereof, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical antipodes and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be for example (+) or (−)-menthol and an optically active acyl group in amides, for example, may be a (+)-or (−)-menthyloxycarbonyl.

Furthermore, the compounds of formula I may be converted into the salts thereof, particularly for pharmaceutical use into the physiologically acceptable salts with inorganic or organic acids. Acids which may be used for this purpose include for example hydrochloric acid, hydrobromic acid, sulphuric acid, phosphoric acid, fumaric acid, succinic acid, lactic acid, citric acid, tartaric acid or maleic acid.

Moreover, if the new compounds of formula I thus obtained contain an acidic group such as a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, arginine, cyclohexylamine, etha-nolamine, diethanolamine and triethanolamine.

The compounds of general formulae II to V used as starting materials are either known from the literature or may be obtained by methods known from the literature or are described in the Examples.

A compound of general formula II is obtained, for example, by reacting a compound of general formula

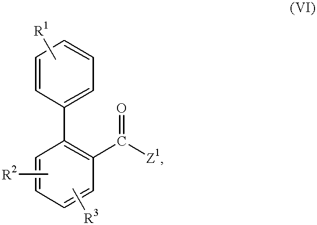

wherein R¹ to R³ are as hereinbefore defined and Z¹ denotes a carboxy group or a reactive derivative of a carboxy group, with an amine of general formula

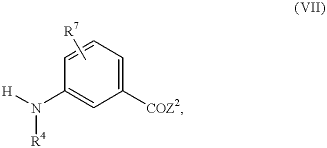

wherein R⁴ to R⁷ are as hereinbefore defined and Z² denotes a protecting group for a carboxy group, and subsequently cleaving the protecting group.

The amines of general formula III wherein R⁶ denotes a heteroaryl-aryl group or a heteroaryl-aryl-C₁₋₆-alkyl group, may be prepared, for example, by synthesising the heteroaromatic ring from suitably substituted aryl or aryl-C₁₋₆-alkyl educts, possibly by reactions of condensation with suitable dicarbonyl compounds.

The biphenyl-2-carboxylic acids according to general formula IV are known from the literature or may be prepared by methods known from the literature from corresponding biphenyl educts.

The 3-amino-benzoic acid amides according to general formula VI are also known from the literature or may easily be prepared from optionally substituted 3-aminobenzoic acids by reacting with the corresponding amines.

As already mentioned hereinbefore, the compounds of general formula I and the physiologically acceptable salts thereof have valuable pharmacological properties. In particular, they are valuable inhibitors of the microsomal triglyceride-transfer protein (MTP) and are therefore suitable for lowering the plasma levels of the atherogenic lip oproteins.

For example, the compounds according to the invention were investigated for their biological effects as follows:

Inhibitors of MTP were identified by a cell-free MTP activity test. Solubilised liver microsomes from various species (e.g. rat, pig) can be used as the MTP source. To prepare the donor and acceptor vesicles, lipids dissolved in organic solvents were mixed in a suitable ratio and applied to the wall of glass container in a thin layer by blowing the solvent in a nitrogen current. The solution used to prepare donor vesicles contained 400 μM of phosphatidyl choline, 75 μM of cardiolipin and 10 μM of [¹⁴C]-triolein (68.8 μCi/mg). To prepare the acceptor vesicles, a solution of 1.2 mM of phosphatidyl choline, 5 μM of triolein and 15 μM of [³H]-dipalmitoyl-phosphatidyl choline (108 mCi/mg) was used. Vesicles are produced by wetting the dried lipids with test buffer and subsequently treating with ultrasound. Vesicle populations of uniform size were obtained by gel filtration of the ultrasound-treated lipids. The MTP activity test contains donor vesicles, acceptor vesicles as well as the MTP source in test buffer. Substances were added from concentrated DMSO-containing stock solutions, the final concentration of DMSO in the test was 0.1%. The reaction was started by the addition of MTP. After a corresponding incubation time the transfer process was stopped by the addition of 500 μl of a SOURCE 30Q anion exchanger suspension (Pharmacia Biotech). The mixture was shaken for 5 minutes and the donor vesicles bound to the anion exchanger material were separated off by centrifuging. The radioactivity of [3H] and [¹⁴C] in the supernatant was determined by liquid scintillation measurement and from this the recovery of the acceptor vesicles and the triglyceride transfer speed was calculated. The compounds of general formula I exhibit IC₅₀ values≦100μM in the test described.

In view of the abovementioned biological properties the compounds of general formula I and the physiologically acceptable salts thereof are particularly suitable for lowering the plasma concentration of atherogenic apolipoprotein B (apoB)-containing lipoproteins such as chylomicrons and/or very low density lipoproteins (VLDL) as well as the residues thereof such as low density lipoproteins (LDL) and/or lipoprotein(a) (Lp(a)), for treating hyperlipidaemias, for preventing and treating atherosclerosis and the clinical sequelae thereof, and for preventing and treating related disorders such as diabetes mellitus, adiposity and pancreatitis, oral administration being preferred.

The daily dose needed to achieve such an effect is between 0.5 and 500 mg, expediently between 1 and 350 mg, but preferably between 5 and 200 mg, in adults.

For this purpose, the compounds of formula I prepared according to the invention, optionally combined with other active substances such as other lipid-lowering agents, for example HMG-CoA-reductase-inhibitors, cholesterol biosynthesis inhibitors such as squalene synthase inhibitors and squalene cyclase inhibitors, bile acid-binding resins, fibrates, cholesterol resorption inhibitors, niacin, probucol, CETP inhibitors and ACAT inhibitors together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethyleneglycol, propylene glycol, stearyl alcohol, carb-oxymethylcellulose or fatty substances such as hard fat or suitable mixtures thereof in conventional galenic preparations such as plain or coated tablets, capsules, powders, suspensions or suppositories.

The Examples which follow are intended to illustrate the invention in more detail:

### EXAMPLE 1

N-[4-(3-methyl-5-phenyl-pyrazol-1-yl)-phenylmethyl]-3-(4′-methylbiphenyl-2-carbonylamino)-benzoic acid amide

a. 4-(3-methyl-5-phenyl-pyrazol-1-yl)-benzonitrile A solution of 20.0 g (0.118 mol) of 4-cyanophenylhydrazine and 19.1 g (0.118 mol) of benzoylacetone in 600 ml methanol is combined with 16.7 ml of triethylamine and stirred for two days. The solvent is distilled off, the residue is distributed in dichloromethane/water and the combined organic extracts are dried. The residue is chromatographed on silica gel, eluting with dichloromethane.

Yield:22.2 g (73% of theory),

R_{f} value:0.9 (silica gel; dichloromethane/methanol=19:1)

C₁₇H₁₃N₃ (259.31)

Mass spectrum:(M+H)⁺=260

b. 4-(3-methyl-5-phenyl-pyrazol-1-yl)-phenylmethylamine

22.2 g (0.086 mol) of 4-(3-methyl-5-phenyl-pyrazol-1-yl)-benzonitrile are dissolved in 660 ml of methanolic ammonia and after the addition of Raney nickel hydrogenated at ambient temperature with hydrogen. The catalyst is filtered off and the solution is evaporated down.

The residue is chromatographed on silica gel, eluting with dichloromethane/methanol=4:1.

Yield:22 g (97% of theory),

R_{f} value:0.2 (silica gel; dichloromethane/methanol=9:1)

C₁₇H₁₇N₃ (263.35)

Mass spectrum:(M+H)⁺=264 M⁺=263

c. ethyl 3-(4′-methylbiphenyl-2-carbonylamino)-benzoate

1.6 g (9.9 mmol) of ethyl 3-aminobenzoate are placed in 80 ml of tetrahydrofuran and 2.8 mol (20 mmol) of triethylamine, a solution of 2.3 g (9.9 mmol) of 4′-methylbiphenyl-2-carboxylic acid chloride is added dropwise and the mixture is stirred for 1 more hour. The solvent is distilled off, the residue is distributed in ethyl acetate/water, the combined organic extracts are dried and evaporated down.

Yield:3.5 g (98% of theory),

R_{f} value:0.7 (silica gel; dichloromethane/methanol=19:1)

d. 3-(4′-methylbiphenyl-2-carbonylamino)-benzoic acid

3.5 g (9.7 mmol) of ethyl 4′-methylbiphenyl-2-carbonylamino)-benzoate are stirred in 100 ml methanol and 15 ml of 2 molar sodium hydroxide solution for 1 hour at 50° C. The solvent is distilled off, the residue is combined with water and acidified with 2 molar hydrochloric acid. Precipitated product is suction filtered.

Yield:3.2 g (99% of theory),

R_{f} value:0.2 (silica gel; dichloromethane/methanol=19:1)

e. 3-(4′-methylbiphenyl-2-carbonylamino)-benzoic acid chloride

490 mg (1.5 mmol) of 4′-methylbiphenyl-2-carbonylamino)-benzoic acid are stirred in 5 ml thionyl chloride with the addition of 3 drops of dimethylformamide for 1 hour. Then the mixture is evaporated down and the residue is further reacted directly.

Yield:518 mg (100% of theory).

f. N-[4-(3-methyl-5-phenyl-pyrazol-1-yl)-phenylmethyl]-3-(4′-methylbiphenyl-2-carbonylamino)-benzoic acid amide

A mixture of 518 mg (1.5 mmol) of 3-(4′-methylbiphenyl-2-carbonylamino)-benzoic acid chloride, 390 mg (1.5 mmol) of 4-(3-methyl-5-phenyl-pyrazol-1-yl)-phenylmethylamine and 0.7 ml (5 mmol) of triethylamine are stirred in 20 ml tetrahydrofuran for 1 hour. The solvent is distilled off and the residue chromatographed on silica gel, eluting with dichloromethane/ethanol 0-4%.

Yield:340 mg (40% of theory),

R_{f} value:0.7 (silica gel; dichloromethane/ethanol=9:1)

C₃₈H₃₂N₄O₂ (576.70)

Mass spectrum:(M+H)⁺=577(M−H)⁻=575(M+Na)⁺=599

### EXAMPLE 2

N-[4-(3-methyl-5-phenyl-pyrazol-1-yl)-phenylmethyl]-3-(4′-trifluoromethylbiphenyl-2-carbonylamino)-benzoic acid amide

Prepared analogously to Example 1f from 3-(4′-trifluoromethyl-biphenyl-2-carbonylamino)-benzoic acid chloride and 4-(3-methyl-5-phenyl-pyrazol-1-yl)-phenylmethylamine in tetrahydrofuran with the addition of triethylamine.

Yield:47% of theory,

R_{f} value:0.5 (silica gel; dichloromethane/ethanol=19:1)

C₃₈H₂₉F₃N₄O₂ (630.67)

Mass spectrum:(M+H)⁺=631(M−H)⁻=629(M+Na)⁺=653

### EXAMPLE 3

N-[4-(3-methyl-5-phenyl-pyrazol-1-yl)-phenylmethyl]-3-(biphenyl-2-carbonylamino)-benzoic acid amide

Prepared analogously to Example if from 3-(biphenyl-2-carbonylamino)-benzoic acid chloride and 4-(3-methyl-5-phenyl-pyrazol-1-yl)-phenylmethylamine in tetrahydrofuran with the addition of triethylamine.

Yield:54% of theory,

R_{f} value:0.4 (silica gel; dichloromethane/ethanol=19:1)

C₃₇H₃₀N₄O₂ (562.67)

Mass spectrum:(M+H)⁺=563(M−H)⁻=561 (M+Na)⁺=585

### EXAMPLE 4

N-[4-(3-methyl-5-phenyl-pyrazol-1-yl)-phenylmethyl]-3-(4′-fluorobiphenyl-2-carbonylamino)-benzoic acid amide

Prepared analogously to Example 1f from 3-(4′-fluorobiphenyl-2-carbonylamino)-benzoic acid chloride and 4-(3-methyl-5-phenyl-pyrazol-1-yl)-phenylmethylamine in tetrahydrofuran with the addition of triethylamine.

Yield:52% of theory,

R_{f} value:0.2 (silica gel; dichloromethane/ethanol=50:1)

C₃₇H₂₉FN₄O₂ (580.66)

Mass spectrum:(M−H)⁻=579 (M+Na)⁺=603

### EXAMPLE 5

N-[4-(N-methyl-N-phenylaminocarbonyl)-phenylmethyl] -3-(4′-trifluoromethylbiphenyl-2-carbonylamino)-benzoic acid amide

Prepared analogously to Example if from 3-(4′-trifluoromethyl-biphenyl-2-carbonylamino)-benzoic acid chloride and 4-amino-N-methyl-N-phenyl-benzoic acid amide in tetrahydrofuran with the addition of triethylamine.

Yield:37% of theory,

R_{f} value:0.5 (silica gel; dichloromethane/ethanol=19:1)

C₃₆H₂₈F₃N₃O₃ (607.64)

Mass spectrum:(M+H)⁺=608 (M−H)⁻=606 (M+Na)⁺=630

### EXAMPLE 6

N-[4-(N-methyl-N-phenylaminocarbonyl)-phenylmethyl]-3-(biphenyl-2-carbonylamino)-benzoic acid amide

Prepared analogously to Example if from 3-(biphenyl-2-carbonylamino)-benzoic acid chloride and 4-amino-N-methyl-N-phenyl-benzoic acid amide in tetrahydrofuran with the addition of triethylamine

Yield:35% of theory,

R_{f} value:0.4 (silica gel; dichloromethane/ethanol=19:1)

C₃₅H₂₉N₃O₃ (539.64)

Mass spectrum:(M+H)⁺=540 (M−H)⁻=538 (M+Na)⁺=562

### EXAMPLE 7

N-(biphenyl-4-methyl)-3-(4′-trifluoromethylbiphenyl-2-carbonylamino)-benzoic acid amide

A solution of 0.3 g (0.8 mmol) of 3-(4′-trifluoromethyl-biphenyl-2-carbonylamino)-benzoic acid, 0.1 g (0.8 mmol) of 4-phenyl-benzylamine and 0.5 ml (4.6 mmol) of N-methylmorpholine in 25 ml of dichloromethane is combined with 0.9 ml (1.6 mmol) of propanephosphonic acid cycloanhydride (50 wt-% in ethyl acetate) at −10° C. and stirred for 2 hours while cooling. The mixture is chromatographed on silica gel, eluting with a gradient from 100%-dichloromethane to dichloromethane/methanol/ammonia=20:77.5:2.5.

Yield:0.2 g (47% of theory),

R_{f} value:0.75 (silica gel; dichloromethane/ethanol=9:1)

C₃₄H₂₅F₃N₂O₂ (550.58)

Mass spectrum:(M−H)⁻=549

### EXAMPLE 8

N-(pyridine-3-yl-methyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 3-picolylamine in dichloromethane with the addition of propanephosphonic acid cycloanhydride and N-methylmorpholine.

Yield:81% of theory

R_{f} value:0.75 (silica gel; dichloromethane/ethanol 9:1)

C₂₇H₂₀F₃N₃O₂ (475.47)

Mass spectrum:(M−H)⁻=474

### EXAMPLE 9

N-(2-phenylethyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 2-phenylethylamine in dichloromethane with the addition of propanephosphonic acid cycloanhydride and N-methylmorpholine.

Yield:60% of theory

R_{f} value:0.72 (silica gel; dichloromethane/ethanol=9:1)

C₂₉H₂₃F₃N₂O₂ (488.51)

Mass spectrum:(M−H)⁻=487

### EXAMPLE 10

N-(4-benzoylamino-phenylmethyl)-3-(biphenyl-2-carbonylamino)-benzoic acid amide

Prepared analogously to Example 7 from 3-(biphenyl-2-carbonylamino)-benzoic acid and 4-benzoylamino-phenylmethylamine in dichloromethane with the addition of propanephosphonic acid cycloanhydride and N-methylmorpholine.

Yield:57% of theory

R_{f} value:0.56 (silica gel; dichloromethane/ethanol=9:1)

C₃₄H₂₇N₃O₃ (525.61)

Mass spectrum:(M−H)−=524

### EXAMPLE 11

N-(2-acetylamino-ethyl)-3-(4′-trifluoromethylbiphenyl-2-carbonylamino)-benzoic acid amide

Prepared analogously to Example 7 from 3-(4′-trifluoromethylbiphenylene-2-carbonylamino)-benzoic acid and N-(2-amino-ethyl)-acetamide in dichloromethane with the addition of propanephosphonic acid cycloanhydride and N-methylmorpholine.

Yield:41% of theory

R_{f} value:0.45 (silica gel; dichloromethane/ethanol=9:1)

C₂₅H₂₂F₃N₃O₃ (469.46)

Mass spectrum:(M−H)⁻=468

### EXAMPLE 12

N-(4-benzoylamino-phenylmethyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 4-benzoylamino-phenylmethylamine in dichloromethane with the addition of propanephosphonic acid cycloanhydride and N-methylmorpholine.

Yield:30% of theory

R_{f} value:0.72 (silica gel; dichloromethane/ethanol=9:1)

C₃₅H₂₆F₃N₃O₃ (593.61)

Mass spectrum:(M−H)⁻=592

### EXAMPLE 13

N-phenyl-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and aniline in dichloromethane with the addition of propanephosphonic acid cycloanhydride and N-methylmorpholine.

Yield:59% of theory

R_{f} value:0.72 (silica gel; dichloromethane/ethanol=9:1)

C₂₇H₁₉F₃N₂O₂ (460.46)

Mass spectrum:(M−H)⁻=459 (M+Na)⁺=483

### EXAMPLE 14

N-methyl-N-propyl-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and N-methyl-propylamine in dichloromethane with the addition of propanephosphonic acid cycloanhydride and N-methylmorpholine.

Yield:44% of theory

R_{f} value:0.73 (silica gel; dichloromethane/ethanol=9:1)

C₂₅H₂₃F₃N₂O₂ (440.47)

Mass spectrum:(M−H)⁻=439

### EXAMPLE 15

N-(2-ethoxycarbonylethyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and β-alanine ethyl ester in dichloromethane with the addition of propanephosphonic acid cycloanhydride and N-methylmorpholine.

Yield:11% of theory

R_{f} value:0.73 (silica gel; dichloromethane/ethanol=9:1)

C₂₆H₂₃F₃N₂O₄ (484.48)

Mass spectrum:(M−H)⁻=483 (M+Na)⁺=507

### EXAMPLE 16

N-tert.butoxycarbonylamino-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and tert.butyl hydrazinoformate in dichloromethane with the addition of propanephosphonic acid cycloanhydride and N-methylmorpholine.

Yield:46% of theory

R_{f} value:0.58 (silica gel; dichloromethane/ethanol=9:1)

C₂₆H₂₄F₃N₃O₄ (499.49)

Mass spectrum:(M−H)⁻=498

### EXAMPLE 17

N-phenylamino-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and phenylhydrazine in dichloromethane with the addition of propanephosphonic acid cycloanhydride and N-methylmorpholine.

Yield:8% of theory

R_{f} value:0.72 (silica gel; dichloromethane/ethanol=9:1)

C₂₇H₂₀F₃N₃O₂ (475.47)

Mass spectrum:(M−H)⁻=474 (M+Na)⁺=498

### EXAMPLE 18

N-(N-tert.butoxycarbonyl-piperidin-4-yl-methyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and N-tert.butoxy-carbonyl-piperidin-4-yl-methylamine in dichloromethane with the addition of propanephosphonic acid cycloanhydride and N-methylmorpholine.

Yield:38% of theory

R_{f} value:0.68 (silica gel; dichloromethane/ethanol=9:1)

C₃₂H₃₄F₃N₃O₄ (581.64)

Mass spectrum:(M−H)⁻=580 (M+Na)⁺=604

### EXAMPLE 19

N-phenylmethyl-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and benzylamine in dichloromethane with the addition of propanephosphonic acid cycloanhydride and N-methylmorpholine.

Yield:46% of theory

R_{f} value:0.68 (silica gel; dichloromethane/ethanol=9:1)

C₂₈H₂₁F₃N₂O₂ (474.49)

Mass spectrum:(M−H)⁻=473 (M+Na)⁺=497

### EXAMPLE 20

N-(biphenyl-2-methyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 2-phenyl-benzylamine in dichloromethane with the addition of propanephosphonic acid cycloanhydride and N-methylmorpholine.

Yield:65% of theory

R_{f} value:0.74 (silica gel; dichloromethane/ethanol=9:1)

C₃₄H₂₅F₃N₂O₂ (550.59)

Mass spectrum:(M−H)⁻=549 (M+Na)⁺=573

### EXAMPLE 21

N-propyl-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and propylamine in dichloromethane with the addition of propanephosphonic acid cycloanhydride and N-methylmorpholine.

Yield:33% of theory

R_{f} value:0.67 (silica gel; dichloromethane/ethanol=9:1)

C₂₄H₂lF₃N₂O₂ (426.44)

Mass spectrum:(M−H)⁻=425 (M+Na)⁺=449

### EXAMPLE 22

N-ethoxycarbonylmethyl-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and glycine ethyl ester hydrochloride in dichloromethane with the addition of propanephosphonic acid cycloanhydride and N-methylmorpholine.

Yield:79% of theory

R_{f} value:0.67 (silica gel; dichloromethane/ethanol=9:1)

C₂₅H₂lF₃N₂O₄ (470.45)

Mass spectrum:(M−H)⁻=469 (M+Na)⁺=493

### EXAMPLE 23

N-dimethylamino-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and N,N-dimethylhydrazine in dichloromethane with the addition of propanephosphonic acid cycloanhydride and N-methylmorpholine.

Yield:57% of theory

R_{f} value:0.85 (silica gel; dichloromethane/ethanol=9:1)

C₂₃H₂₀F₃N₃O₂ (427.43)

Mass spectrum:(M−H)⁻=426

(M+H)⁺=428 (M+Na)⁺=450

### Example 24

N-phenylmethyl-N-methyl-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and N-methyl-benzylamine in dichloromethane with the addition of propanephosphonic acid cycloanhydride and N-methylmorpholine.

Yield:95% of theory

R_{f} value:0.72 (silica gel; dichloromethane/ethanol=9:1)

C₂₉H₂₃F₃N₂O₂ (488.51)

Mass spectrum:(M−H)⁻=487 (M+Na)⁺=511

### EXAMPLE 25

N-[4-(phenylmethyl)-phenyl]-3-(4-methylbiphenyl-2-carbonylamino)-benzoic acid amide

Prepared analogously to Example if from 4′-methylbiphenyl-2-carboxylic acid chloride and 3-amino-N-(4-benzyl-phenyl)-benzoic acid amide in tetrahydrofuran with the addition of triethylamine.

Yield:83% of theory,

R_{f} value:0.6 (silica gel; dichloromethane/ethanol=9:1)

C₃₄H₂₈N₂O₂ (496.61)

Mass spectrum:(M−H)⁻=495

### EXAMPLE 26

N-(biphenyl-3-methyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 3-phenylbenzylamine in dichloromethane with the addition of propanephosphonic acid cycloanhydride and N-methylmorpholine.

Yield:58% of theory

R_{f} value:0.71 (silica gel; dichloromethane/ethanol=9:1) C₃₄H₂₅F₃N₂O₂ (550.59)

Mass spectrum:(M−H)⁻=549 (M+Na)⁺=573

### EXAMPLE 27

N-[4-(1H-imidazol-2-yl)-phenylmethyl] -3-(biphenyl-2-carbonylamino)-benzoic acid amide-hydrochloride

Prepared analogously to Example 7 from 3-(biphenyl-2-carbonyl-amino)-benzoic acid and 4-(1H-imidazol-2-yl)benzylamine in dichloromethane with the addition of propanephosphonic acid cycloanhydride and N-methylmorpholine.

Yield:96% of theory

R_{f} value:0.5 (silica gel; dichloromethane/ethanol=95:5)

C₃₀H₂₄N₄O₂ ×HCl (472.54/509.01)

Mass spectrum:(M+H)⁺=473

### EXAMPLE 28

N-(biphenyl-4-methyl)-3-(biphenyl-2-carbonylamino)-benzoic acid amide

Prepared analogously to Example 7 from 3-(biphenyl-2-carbonyl-amino)-benzoic acid and 4-phenylbenzylamine in dichloromethane with the addition of propanephosphonic acid cycloanhydride and N-methylmorpholine.

Yield:88% of theory

R_{f} value:0.76 (silica gel; dichloromethane/ethanol=95:5)

C₃₃H₂₆N₂O₂ (482.59)

Mass spectrum:(M−H)⁻=481 (M+H)⁺=483 (M+Na)⁺=505

### EXAMPLE 29

N-(4′-hydroxybiphenyl-4-methyl)-3-(biphenyl-2-carbonylamino)-benzoic acid amide

Prepared analogously to Example 7 from 3-(biphenyl-2-carbonylamino)-benzoic acid and 4-(4-hydroxyphenyl)-benzylamine in dichloromethane with the addition of propanephosphonic acid cycloanhydride and N-methylmorpholine.

Yield:6% of theory

R_{f} value:0.88 (silica gel; dichloromethane/ethanol=95:5)

C₃₃H₂₆N₂O₃ (498.59)

Mass spectrum:(M−H)⁻=497 (M+Cl)⁻=533/35 (chlorine isotopes)

### EXAMPLE 30

N-(piperidin-4-yl-methyl)-3-(4′-trifluoromethylbiphenyl-2-carbonylamino)-benzoic acid amide-trifluoroacetate

0.2 g (0.27 mmol) of N-(N-tert.butoxycarbonyl-piperidin-4-yl-methyl)-3-(4′-trifluoromethylbiphenyl-2-carbonylamino)-benzoic acid amide are stirred in 30 ml dichloromethane and 3 ml of trifluoroacetic acid for 17 hours at ambient temperature. Then the mixture is evaporated to dryness in vacuo.

Yield:0.2 g (98% of theory),

R_{f} value:0.42 (silica gel; dichloromethane/ethanol=9:1)

C₂₇H₂₆F₃N₃O₂ ×CF₃COOH (481.52/595.55)

Mass spectrum:(M+H)⁺=482

### EXAMPLE 31

N-[N-(N-methyl-N-phenylaminocarbonyl)-piperidin-4-yl-methyl]-3-(4′-trifluoromethylbiphenyl-2-carbonylamino)-benzoic acid amide

Prepared analogously to Example if from N-(piperidin-4-yl-methyl)-3-(4′-trifluoromethylbiphenyl-2-carbonylamino)-benzoic acid amide-trifluoroacetate and N-methyl-N-phenyl-carbamoylchloride in tetrahydrofuran with the addition of triethylamine.

Yield:99% of theory,

R_{f} value:0.57 (silica gel; dichloromethane/ethanol 9:1)

C₃₅H₃₃F₃N₄O₃ (614.67)

Mass spectrum:(M−H)⁻=613

### EXAMPLE 32

N-[4-(3-methyl-5-tert.butyl-pyrazol-1-yl)-phenylmethyl]-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 4-(5-tert.-butyl-3-methyl-pyrazol-1-yl)-benzylamine in dichloromethane with the addition of propanephosphonic acid cycloanhydride and N-methylmorpholine.

Yield:53% of theory

R_{f} value:0.5 (silica gel; dichloromethane/ethanol=19:1)

C₃₆H₃₃F₃N₄O₂ (610.69)

Mass spectrum:(M−H)⁻=609 (M+H)⁺=611 (M+Na)⁺=633

### EXAMPLE 33

N-methyl-N-[4-(3-methyl-5-phenyl-pyrazol-1-yl)-phenylmethyl] -3-(4′-methylbiphenyl-2-carbonylamino)-benzoic acid amide

Prepared analogously to Example 1c from 4′-methylbiphenyl-2-carboxylic acid chloride and N-methyl-N-[4-(3-methyl-5-phenyl-pyrazol-1-yl)-phenylmethyl]-3-amino-benzoic acid amide in tetrahydrofuran with the addition of triethylamine.

Yield:22% of theory

R_{f} value:0.6 (silica gel; dichloromethane/methanol=9:1)

C₃₉H₃₄N₄O₂ (590.73)

Mass spectrum:(M−H)⁻=589 (M+H)⁺=591

### EXAMPLE 34

N-(pyridine-3-yl-methyl)-3-(biphenyl-2-carbonylamino)-benzoic acid amide

3.2 mg (10 mol) of 3-(biphenyl-2-carbonylamino)-benzoic acid are placed in 0.4 ml dimethylformamide and after the addition of 1.6 mg (15 μmol) of 3-picolylamine, 3.9 mg (12 μmol) of O-(benzo-triazol-1-yl)-N,N,N′,N′-tetramethyluronium tetrafluoroborate (TBTU) and 7 mg (50 μmol) of N-ethyl-diisopropylamine the mixture is stirred for 12 hours. The solution is evaporated down.

R_{f} value:0.2 (silica gel; dichloromethane/ethanol=19:1)

C₂₆H₂₁N₃O₂ (407.47)

Mass spectrum:(M+H)⁺=408

### EXAMPLE 35

N-phenyl-3-(biphenyl-2-carbonylamino)-benzoic acid amide

Prepared analogously to Example 34 from 3-(biphenyl-2-carbonylamino)-benzoic acid, aniline, TBTU and N-ethyldiisopropylamine in dimethylformamide.

R_{f} value:0.75 (silica gel; dichloromethane/ethanol=19:1)

C₂₆H₂₀N₂O₂ (392.46)

Mass spectrum:(M+Na)⁺=415

### EXAMPLE 36

N-tert.butyl-3-(biphenyl-2-carbonylamino)-benzoic acid amide

Prepared analogously to Example 34 from 3-(biphenyl-2-carbonyl-amino)-benzoic acid, tert.butylamine, TBTU and N-ethyldiisopropylamine in dimethylformamide.

R_{f} value:0.4 (silica gel; dichloromethane/ethanol=19:1)

C₂₄H₂₄N₂O₂ (372.47)

Mass spectrum:(M+Na)⁺=395

### EXAMPLE 37

N-hydroxyethyl-3-(biphenyl-2-carbonylamino)-benzoic acid amide

Prepared analogously to Example 34 from 3-(biphenyl-2-carbonylamino)-benzoic acid, 2-aminoethanol, TBTU and N-ethyl-diisopropylamine in dimethylformamide.

R_{f} value:0.2 (silica gel; dichloromethane/ethanol=19:1)

C₂₂H₂₀N₂O₃ (360.41)

Mass spectrum:(M+Na)⁺=383

### EXAMPLE 38

N-(2-dimethylamino-ethyl)-3-(biphenyl-2-carbonylamino)-benzoic acid amide

Prepared analogously to Example 34 from 3-(biphenyl-2-carbonylamino)-benzoic acid, N,N-dimethylethylenediamine, TBTU and N-ethyldiisopropylamine in dimethylformamide.

R_{f} value:0.15 (silica gel; dichloromethane/ethanol=4:1)

C₂₄H₂₅N₃O₂ (387.48)

Mass spectrum:(M+H)⁺=388 M⁺=387

### EXAMPLE 39

N-(2-carboxy-ethyl)-3-(biphenyl-2-carbonylamino)-benzoic acid amide-sodium salt

Prepared analogously to Example 34 from 3-(biphenyl-2-carbonylamino)-benzoic acid, β-alanine, TBTU, sodium hydroxide solution and N-ethyldiisopropylamine in dimethylformamide.

R_{f} value:0.15 (silica gel; dichloromethane/ethanol=9:1)

C₂₃H₁₉NaN₂O₄ (410.41), free acid

C₂₃H₂₀N₂O₄ (388.42)

Mass spectrum:(M−H)⁻=387

### EXAMPLE 40

N-(4-[1,2,3]-thiadiazol-4-yl-phenylmethyl)-3-(4′-trifluoro-methylbiphenyl-2-carbonylamino)-benzoic acid amide

Prepared analogously to Example 7 from 3-(4′-trifluoromethylbiphenyl-2-carbonylamino)-benzoic acid and 4-[1,2,3]-thiadiazol-4-yl-benzylamine in dichloromethane with the addition of propanephosphonic acid cycloanhydride and N-methylmorpholine.

Yield:18% of theory

R_{f} value:0.75 (silica gel; dichloromethane/ethanol=9:1)

C₃₀H₂₁F₃N₄O₂S (558.58)

Mass spectrum:(M−H)⁻=557 (M+H)⁺=559 (M+Na)⁺=581

### EXAMPLE 41

N-(4-phenylaminosulphonyl-phenylmethyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 4-aminomethyl-N-phenyl-benzenesulphonamide in dichloromethane with the addition of propanephosphonic acid cycloanhydride and N-methylmorpholine.

Yield:73% of theory

R_{f} value:0.68 (silica gel; dichloromethane/ethanol=9:1) C₃₄H₂₆F₃N₃O₄S (629.66)

Mass spectrum:(M−H)⁻=628 (M+H)⁺=630 (M+Na)⁺=652

### EXAMPLE 42

N-(4-piperidin-1-yl-phenylmethyl)-3-(4′-trifluoromethyl-biphenyl-2-carbonylamino)-benzoic acid amide

Prepared analogously to Example 7 from 3-(4′-trifluoromethylbiphenyl-2-carbonylamino)-benzoic acid and 4-piperidin-1-yl-benzylamine in dichloromethane with the addition of propanephosphonic acid cycloanhydride and N-methylmorpholine.

Yield:47% of theory

R_{f} value:0.69 (silica gel; dichloromethane/ethanol=9:1)

C₃₃H₃₀F₃N₃O₂ (557.61)

Mass spectrum:(M−H)⁻=556 (M+Na)⁺=580

### EXAMPLE 43

N-(4-phenylsulphonylamino-phenylmethyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 4-phenylsulphonylamino-benzylamine in dichloromethane with the addition of propanephosphonic acid cycloanhydride and N-methylmorpholine.

Yield:57% of theory

R_{f} value:0.67 (silica gel; dichloromethane/ethanol=9:1)

C₃₄H₂₆F₃N₃O₄S (629.66)

Mass spectrum:(M−H)⁻=628 (M+H)⁺=630 (M+Na)⁺=652

### EXAMPLE 44

N-[4-(2-methyl-pyrrol-1-yl)-phenylmethyl]-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 4-(2-methyl-pyrrol-1-yl)-benzylamine in dichloromethane with the addition of propanephosphonic acid cycloanhydride and N-methylmorpholine.

Yield:22% of theory

R_{f} value:0.73 (silica gel; dichloromethane/ethanol=9:1)

C₃₃H₂₆F₃N₃O₂ (553.58)

Mass spectrum:(M−H)⁻=552 (M+H)⁺=554 (M+Na)⁺=576

### EXAMPLE 45

N-(2′-methylbiphenyl-4-methyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 4-(2-methylphenyl)benzylamine in dichloromethane with the addition of propanephosphonic acid cycloanhydride and N-methylmorpholine.

Yield:21% of theory

R_{f} value:0.72 (silica gel; dichloromethane/ethanol=9:1)

C₃₅H₂₇F₃N₂O₂ (564.60)

Mass spectrum:(M−H)⁻=563 (M+Na)⁺=587

### EXAMPLE 46

N-(4-tert.butyl-phenylmethyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 4-tert.butyl-benzylamine in dichloromethane with the addition of propanephosphonic acid cycloanhydride and N-methylmorpholine.

Yield:53% of theory

R_{f} value:0.69 (silica gel; dichloromethane/ethanol=9:1)

C₃₂H₂₉F₃N₂O₂ (530.59)

Mass spectrum:(M−H)⁻=529 (M+Na)⁺=553

### EXAMPLE 47

N-(4-isopropyl-phenylmethyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 4-isopropylbenzylamine in dichloromethane with the addition of propanephosphonic acid cycloanhydride and N-methylmorpholine.

Yield:58% of theory

R_{f} value:0.67 (silica gel; dichloromethane/ethanol=9:1)

C₃lH₂₇F₃N₂O₂ (516.56)

Mass spectrum:(M−H)⁻=515 (M+Na)⁺=539

### EXAMPLE 48

N-(4-Bromophenylmethyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 4-bromobenzylamine in dichloromethane with the addition of propanephosphonic acid cycloanhydride and N-methylmorpholine.

Yield:51% of theory

R_{f} value:0.64 (silica gel; dichloromethane/ethanol=9:1)

C₂₈H₂₀BrF₃N₂O₂ (553.38)

Mass spectrum:(M−H)⁻=551/53 (bromine isotopes) (M+Na)⁺=575/77 (bromine isotopes)

### EXAMPLE 49

N-(4-trifluoromethyl-phenylmethyl)-3-(4′-trifluoromethyl-biphenyl-2-carbonylamino)-benzoic acid amide

Prepared analogously to Example 7 from 3-(4′-trifluoromethylbiphenyl-2-carbonylamino)-benzoic acid and 4-trifluoromethyl-benzylamine in dichloromethane with the addition of propanephosphonic acid cycloanhydride and N-methylmorpholine.

Yield:48% of theory

R_{f} value:0.63 (silica gel; dichloromethane/ethanol=9:1)

C₂₉H₂₀F₆N₂O₂ (542.48)

Mass spectrum:(M−H)⁻=541 (M+Na)⁺=565

### EXAMPLE 50

N-(4-acetylamino-phenylmethyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 4-acetylaminobenzylamine in dichloromethane with the addition of propanephosphonic acid cycloanhydride and N-methylmorpholine.

Yield:38% of theory

R_{f} value:0.60 (silica gel; dichloromethane/ethanol=9:1)

C₃₀H₂₄F₃N₃O₃ (531.53)

Mass spectrum:(M+Na)⁺=554

### EXAMPLE 51

N-(1H-benzimidazol-2-yl-methyl)-3-(4′-trifluoromethyl-biphenyl-2-carbonylamino)-benzoic acid amide

Prepared analogously to Example 7 from 3-(4′-trifluoromethylbiphenyl-2-carbonylamino)-benzoic acid and 2-(aminomethyl)-benzimidazole in dichloromethane with the addition of propanephosphonic acid cycloanhydride and N-methylmorpholine.

Yield:19% of theory

R_{f} value:0.58 (silica gel; dichloromethane/ethanol=9:1)

C₂₉H₂₁F₃N₄O₂ (514.51)

Mass spectrum:(M−H)⁻=513 (M+H)⁺=515

### EXAMPLE 52

N-(4′-methylbiphenyl-4-methyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 4-(4′-methylphenyl)-benzylamine in dichloromethane with the addition of propanephosphonic acid cycloanhydride and N-methylmorpholine.

Yield:21% of theory

R_{f} value:0.73 (silica gel; dichloromethane/ethanol=9:1)

C₃₅H₂₇F₃N₂O₂ (564.61) Mass spectrum:(M−H)⁻=563 (M+Na)⁺=587

### EXAMPLE 53

N-(4-methyl-phenylmethyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 4-methylbenzylamine in dichloromethane with the addition of propanephosphonic acid cycloanhydride and N-methylmorpholine.

Yield:82% of theory

R_{f} value:0.75 (silica gel; dichloromethane/ethanol=9:1)

C₂₉H₂₃F₃N₂O₂ (488.51)

Mass spectrum:(M−H)⁻=487 (M+Na)⁺=511

### EXAMPLE 54

N-(biphenyl-4-methyl)-2-methyl-5-(biphenyl-2-carbonylamino)-benzoic acid amide

Prepared analogously to Example Ic from biphenyl-2-carboxylic acid chloride and N-(biphenyl-4-methyl)-2-methyl-5-amino-benzoic acid amide in tetrahydrofuran and triethylamine.

Yield:92% of theory

R_{f} value:0.74 (silica gel; dichloromethane/ethanol=9:1)

C₃₄H₂₈N₂O₂ (496.61)

Mass spectrum:(M−H)⁻=495 (M+Na)⁺=519

### EXAMPLE 55

N-(biphenyl-4-methyl)-4-methyl-3-(biphenyl-2-carbonylamino)-benzoic acid amide

Prepared analogously to Example 7 from 3-(biphenyl-2-carbonylamino)-4-methyl-benzoic acid and biphenyl-4-methylamine in dichloromethane with the addition of propanephosphonic acid cycloanhydride and N-methylmorpholine.

Yield:30% of theory

R_{f} value:0.73 (silica gel; dichloromethane/ethanol=9:1)

C₃₄H₂₈N₂O₂ (496.61)

Mass spectrum:(M−H)⁻=495

### EXAMPLE 56

N-(Naphthalin-2-yl-methyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and naphthalin-2-yl-methylamine in dichloromethane with the addition of propanephosphonic acid cycloanhydride and N-methylmorpholine.

Yield:50% of theory

R_{f} value:0.73 (silica gel; dichloromethane/ethanol=9:1)

C₃₂H₂₃F₃N₂O₂ (524.54)

Mass spectrum:(M−H)⁻=523 (M+H)⁺=525 (M+Na)⁺=547

### EXAMPLE 57

N-[4-(N-Methyl-N-cyclohexyl-aminocarbonyl)-phenylmethyl] -3- (biphenyl-2-carbonylamino)-benzoic acid amide

Prepared analogously to Example 7 from 3-(biphenyl-2-carbonylamino)-benzoic acid and 4-(N-methyl-N-cyclohexyl-aminocarbonyl)-phenylmethylamine in dichloromethane with the addition of propanephosphonic acid cycloanhydride and N-methylmorpholine.

Yield:72% of theory

R_{f} value:0.61 (silica gel; dichloromethane/ethanol=9:1)

C₃₅H₃₅N₃O₃ (545.68)

Mass spectrum:(M−H)⁻=544 (M+Na)⁺=568

### EXAMPLE 58

N-[4-(N-methyl-N-phenylcarbonyl-amino)-phenylmethyl]-3- (biphenyl-2-carbonylamino)-benzoic acid amide

Prepared analogously to Example 7 from 3-(biphenyl-2-carbonylamino)-benzoic acid and 4-(N-methyl-N-phenylcarbonyl-amino)-phenylmethylamine in dichloromethane with the addition of propanephosphonic acid cycloanhydride and N-methylmorpholine.

Yield:37% of theory

R_{f} value:0.59 (silica gel; dichloromethane/ethanol=9:1)

C₃₅H₂₉N₃O₃ (539.64)

Mass spectrum:(M−H)⁻=538 (M+H)⁺=540 (M+Na)⁺=562

### EXAMPLE 59

N-(4-bromo-phenylmethyl)-3-(biphenyl-2-carbonylamino)-benzoic acid amide

Prepared analogously to Example 34 from 3-(biphenyl-2-carbonylamino)-benzoic acid, 4-bromo-benzylamine, TBTU and N-ethyldiisopropylamine in dimethylformamide.

Yield:100% of theory

R_{f} value:0.75 (silica gel; dichloromethane/ethanol=9:1)

C₂₇H₂₁BrN₂O₂ (485.38)

Mass spectrum:(M+Na)⁺=507/509 (bromine isotopes)

EXAMPLE 60

N-(1H-Benzimidazol-5-ylmethyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and (1H-benzimidazol-5-yl)-methylamine in dichloromethane with the addition of propanephosphonic acid cycloanhydride and N-methylmorpholine.

Yield:93% of theory

R_{f} value:0.65 (silica gel; dichloromethane/ethanol=9:1)

C₂₉H2lF₃N₄O₂ (514.51)

Mass spectrum:(M−H)⁻=513 (M+Na)⁺=537

### EXAMPLE 61

N-(4′-methylbiphenyl-4-methyl)-3-(biphenyl-2-carbonylamino)-benzoic acid amide

Prepared analogously to Example 7 from 3-(biphenyl-2-carbonylamino)-benzoic acid and 4-(4-methylphenyl)-benzylamine in dichloromethane with the addition of propanephosphonic acid cycloanhydride and N-methylmorpholine.

Yield:47% of theory

R_{f} value:0.7 (silica gel; dichloromethane/ethanol=9:1)

C₃₄H₂₈N₂O₂ (496.61)

Mass spectrum:(M−H)⁻=495 (M+Na)⁺=519

### EXAMPLE 62

N-(2′-tert.Butoxycarbonylbiphenyl-4-methyl)-3-(4′-trifluoromethylbiphenyl-2-carbonyl-amino)-benzoic acid amide

Prepared analogously to Example 7 from 3-(4′-trifluoromethylbiphenyl-2-carbonylamino)-benzoic acid and 4-(2-tert.butoxyphenyl)benzylamine in dichloromethane with the addition of propanephosphonic acid cycloanhydride and N-methylmorpholine.

Yield:46% of theory

R_{f} value:0.81 (silica gel; dichloromethane/ethanol=9:1)

C₃₉H₃₃F₃N₂O₄ (650.70)

Mass spectrum:(M−H)⁻=649 (M+Na)⁺=673

### EXAMPLE 63

N-(2′-Hydroxycarbonylbiphenyl-4-methyl)-3-(4′-trifluoromethylbiphenyl-2-carbonyl-amino)-benzoic acid amide

Prepared analogously to Example 30 from N-(2′-tert.butoxycarbonylbiphenyl-4-methyl)-3-(4′-trifluoromethylbiphenyl-2-carbonylamino)-benzoic acid amide and trifluoroacetic acid in dichloromethane.

Yield:95% of theory

R_{f} value:0.64 (silica gel; dichloromethane/ethanol=9:1)

C₃₅H2₅F₃N₂O₄ (594.59)

Mass spectrum:(M−H)⁻593

### EXAMPLE 64

N-(4-Aminophenyl)methyl-3-(4′-trifluoromethylbiphenyl-2-carbonylamino)-benzoic acid amide-hydrochloride

Prepared analogously to Example 34 from 3-(4′-trifluoromethylbiphenyl-2-carbonylamino)-benzoic acid, 4-amino-benzylamine, TBTU and N-ethyl-diisopropylamine in dimethylformamide and subsequent treatment with dilute HCl.

Yield:24% of theory

R_{f} value:0.28 (silica gel; dichloromethane/ethanol=9.5:0.5)

C₂₈H₂₂F₃N₃O₂ ×HCl(489.50/525.96)

Mass spectrum:(M−H)⁻=488 (M+Na)⁺=512 (M+Cl)⁻=524/26 (chlorine isotopes)

### EXAMPLE 65

N-[4-(N-methyl-N-cyclohexylcarbonyl-amino)-phenylmethyl]-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 4-(N-methyl-N-cyclohexylcarbonyl-amino)-benzylamine in dichloromethane with the addition of propanephosphonic acid cycloanhydride and N-methylmorpholine.

Yield:49% of theory

R_{f} value:0.72 (silica gel; dichloromethane/ethanol=9:1)

C₃₆H₃₄F₃N₃O₃ (613.68)

Mass spectrum:(M−H)⁻=612

### EXAMPLE 66

N-(1-Phenyl-piperidin-4-yl-methyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and (1-phenyl-piperidin-4-yl)-methylamine in dichloromethane with the addition of propanephosphonic acid cycloanhydride and N-methylmorpholine.

Yield:50% of theory

R_{f} value:0.68 (silica gel; dichloromethane/ethanol=9:1)

C₃₃H₃₀F₃N₃O₂ (557.62)

Mass spectrum:(M−H)⁻=556 (M+Na)⁺=580

### EXAMPLE 67

N-[3-methyl-4-(phenylcarbonylamino)-phenylmethyl] -3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 3-methyl-4-(phenylcarbonylamino)-benzylamine in dichloromethane with the addition of propanephosphonic acid cycloanhydride and N-methylmorpholine.

Yield:89% of theory

R_{f} value:0.66 (silica gel; dichloromethane/ethanol=9:1)

C₃₆H₂₈F₃N₃O₃ (607.63)

Mass spectrum:(M−H)⁻=606

### EXAMPLE 68

N-(4-Cyclohexylcarbonylamino-phenylmethyl)-3-(4′-trifluoromethylbiphenyl-2-carbonylamino)-benzoic acid amide

Prepared analogously to Example if from N-(4-amino-phenylmethyl)-3-(4′-trifluoromethylbiphenyl-2-carbonylamino)-benzoic acid amide and cyclohexanecarboxylic acid chloride in tetrahydrofuran with the addition of triethylamine.

Yield:6% of theory

R_{f} value:0.87 (silica gel; dichloromethane/ethanol=4:1)

C₃₅H₃₂F₃N₃O₃ (599.65)

Mass spectrum:(M−H)⁻=598

### EXAMPLE 69

N-(4-tert.butoxycarbonylamino-phenylmethyl)-3-(4′-trifluoromethylbiphenyl-2-carbonylamino)-benzoic acid amide

Prepared analogously to Example if from N-(4-amino-phenylmethyl)-3-(4′-trifluoromethylbiphenyl-2-carbonylamino)-benzoic acid amide and pivalic acid chloride in tetrahydrofuran with the addition of triethylamine.

Yield:46% of theory

R_{f} value:0.78 (silica gel; dichloromethane/ethanol=4:1)

C₃₃H₃₀F₃N₃O₃ (573.62)

Mass spectrum:(M−H)⁻=572

### EXAMPLE 70

N-(naphthalin-1-yl-methyl)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 1-aminomethyl-naphthaline in dichloromethane with the addition of propanephosphonic acid cycloanhydride and N-methylmorpholine.

Yield:53% of theory

R_{f} value:0.70 (silica gel; dichloromethane/ethanol=9:1)

C₃₂H₂₃F₃N₂O₂ (524.54)

Mass spectrum:(M−H)⁻=523 (M+Na)⁺=547

### EXAMPLE 71

N-(3-phenyl-prop-2-in-ylamino)-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and 3-phenyl-prop-2-ynylamine in dichloromethane with the addition of propanephosphonic acid cycloanhydride and N-methylmorpholine.

Yield:54% of theory

R_{f} value:0.72 (silica gel; dichloromethane/ethanol=9:1)

C₃₀H₂lF₃N₂O₂ (498.51)

Mass spectrum:(M−H)⁻=497 (M+Na)⁺=521

### EXAMPLE 72

N-(biphenyl-4-methyl)-N-methyl-3-(4′-trifluoromethylbiphenyl-2-carbonylamino)-benzoic acid amide

Prepared analogously to Example 7 from 3-(4′-trifluoromethylbiphenyl-2-carbonylamino)-benzoic acid and N-methyl-4-phenylbenzylamine in dichloromethane with the addition of propanephosphonic acid cycloanhydride and N-methylmorpholine.

Yield:92% of theory

R_{f} value:0.70 (silica gel; dichloromethane/ethanol=9:1)

C₃₅H₂₇F₃N₂O₂ (564.61)

Mass spectrum:(M−H)⁻563

### EXAMPLE 73

N-(biphenyl-4-methyl)-3-(6-methylbiphenyl-2-carbonylamino)-benzoic acid amide

Prepared analogously to Example 7 from 6-methyl-biphenyl-2-carboxylic acid and N-(biphenyl-4-methyl)-3-amino-benzoic acid amide in dichloromethane with the addition of propanephosphonic acid cycloanhydride and N-methylmorpholine.

Yield:19% of theory

R_{f} value:0.75 (silica gel; dichloromethane/ethanol=9:1)

C₃₄H₂₈N₂O₂ (496.61)

Mass spectrum:(M−H)⁻=495 (M+Na)⁺=519

### EXAMPLE 74

N-[4-(Pyridin-3-yl-carbonylamino)-phenylmethyl]-3-(4′-trifluoromethylbiphenyl-2-carbonylamino)-benzoic acid amide

Prepared analogously to Example 1f from N-(4-amino-phenylmethyl)-3-(4′-trifluoromethylbiphenyl-2-carbonylamino)-benzoic acid amide and nicotinic acid chloride in tetrahydrofuran with the addition of triethylamine.

Yield:39% of theory

R_{f} value:0.72 (silica gel; dichloromethane/ethanol=4:1)

C₃₄H₂₅F₃N₄O₃ (594.59)

Mass spectrum:(M−H)⁻=593

### EXAMPLE 75

N-(4-Butylcarbonylamino-phenylmethyl)-3-(4′-trifluoromethylbi-phenyl-2-carbonylamino)-benzoic acid amide

Prepared analogously to Example if from N-(4-amino-phenylmethyl)-3-(4′-trifluoromethylbiphenyl-2-carbonylamino)-benzoic acid amide and valeric acid chloride in tetrahydrofuran with the addition of triethylamine.

Yield:79% of theory

R_{f} value:0.77 (silica gel; dichloromethane/ethanol=4:1)

C₃₃H₃₀F₃N₃O₃ (573.62)

Mass spectrum:(M−H)⁻=572 (M+Na)⁺=596

### EXAMPLE 76

N-(4-Dimethylamino-phenylmethyl)-3-(biphenyl-2-carbonylamino)-benzoic acid amide

Prepared analogously to Example 34 from 3-(biphenyl-2-carbonylamino)-benzoic acid, 4-dimethylamino-benzylamine, TBTU and N-ethyldiisopropylamine in dimethylformamide.

Yield:100%of theory

R_{f} value:0.35 (silica gel; dichloromethane/ethanol=19:1)

C₂₉H₂₇N₃O₂ (449.55)

Mass spectrum:(M−H)⁻=448 (M+Na)⁺=472

### EXAMPLE 77

N-[4-(Pyridin-4-yl-carbonylamino)-phenylmethyl]-3-(4′-trifluoromethylbiphenyl-2-carbonylamino)-benzoic acid amide

Prepared analogously to Example 1f from N-(4-amino-phenylmethyl)-3-(4′-trifluoromethylbiphenyl-2-carbonylamino)-benzoic acid amide and isonicotinic acid chloride in tetrahydrofuran with the addition of triethylamine.

Yield:40% of theory,

R_{f} value:0.78 (silica gel; dichloromethane/ethanol=4:1)

C₃₄H₂₅F₃N₄0₃ (594.59)

Mass spectrum:(M−H)⁻=593 (M+H)⁺=595

### EXAMPLE 78

N-(2′-methylaminocarbonylbiphenyl-4-methyl)-3-(4′-trifluoromethylbiphenyl-2-carbonylamino)-benzoic acid amide

Prepared analogously to Example 7 from N-(2′-hydroxycarbonylbiphenyl-4-methyl)-3-(4′-trifluoromethylbiphenyl-2-carbonylamino)-benzoic acid amide and methylamine in dichloromethane with the addition of propanephosphonic acid cycloanhydride and N-methylmorpholine.

Yield:7% of theory

R_{f} value:0.43 (silica gel; dichloromethane/ethanol=95:5)

C₃₆H₂₈F₃N₃O₃ (607.64)

Mass spectrum:(M−H)⁻=606 (M+Na)⁺=630

### EXAMPLE 79

N-[4-(Pyrrolidin-1-yl-carbonylamino)-phenylmethyl-3-(4′-trifluoromethylbiphenyl-2-carbonylamino)-benzoic acid amide

Prepared analogously to Example if from N-(4-amino-phenylmethyl)-3-(4′-trifluoromethylbiphenyl-2-carbonylamino)-benzoic acid amide and pyrrolidin-1-carboxylic acid chloride in tetrahydrofuran with the addition of triethylamine.

Yield:81% of theory,

R_{f} value:0.45 (silica gel; dichloromethane/ethanol=9:1)

C₃₃H₂₉F₃N₄0₃ (586.62)

Mass spectrum:(M−H)⁻585

### EXAMPLE 80

N-[4-(4-methyl-piperazin-1-yl)-phenylmethyl]-3-(biphenyl-2-carbonylamino)-benzoic acid amide

Prepared analogously to Example 34 from 3-(biphenyl-2-carbonylamino)-benzoic acid, 4-(4-methyl-piperazin-1-yl)-benzylamine, TBTU and N-ethyldiisopropylamine in dimethylformamide.

R_{f} value:0.20 (silica gel; dichloromethane/ethanol=9:1)

C₃₂H₃₂N₄0₂ (504.63)

Mass spectrum:(M−H)⁻=503 (M+H)⁺=505 (M+Na)⁺=527

### EXAMPLE 81

N-(4-phenylcarbonylamino-phenylmethyl)-3-(6-methylbiphenyl-2-carbonylamino)-benzoic acid amide

Prepared analogously to Example 1 f from 6-methyl-biphenyl-2-carboxylic acid chloride and N-(4-phenylcarbonylamino-phenylmethyl)-3-amino-benzoic acid amide in tetrahydrofuran with the addition of triethylamine.

Yield:85% of theory

R_{f} value:0.40 (silica gel; dichloromethane/ethanol=19:1)

C₃₅H₂₉N₃O₃ (539.64)

Mass spectrum:(M−H)⁻=538 (M+Na)⁺=562

### EXAMPLE 82

N-[4-(pyrrolidin-1-yl)-phenylmethyl]-3-(biphenyl-2-carbonylamino)-benzoic acid amide

Prepared analogously to Example 34 from 3-(biphenyl-2-carbonylamino)-benzoic acid, 4-(pyrrolidin-1-yl)-phenylmethylamine, TBTU and N-ethyldiisopropylamine in dimethylformamide.

R_{f} value:0.70 (silica gel; dichloromethane/ethanol=9:1)

C₃₁H₂₉N₃O₂ (475.59)

Mass spectrum:(M−H)⁻=474 (M+H)⁺=476 (M+Na)⁺=498

### EXAMPLE 83

N-[4-(2-methyl-phenylcarbonylamino)-phenylmethyl]-3-(4′-trifluoromethylbiphenyl-2-carbonylamino)-benzoic acid amide

Prepared analogously to Example if from N-(4-aminophenylmethyl)-3-(4′-trifluoromethylbiphenyl-2-carbonylamino)-benzoic acid amide and 2-tolylic acid chloride in tetrahydrofuran with the addition of triethylamine.

Yield:71% of theory

R_{f} value:0.55 (silica gel; dichloromethane/ethanol=9:1)

C₃₆H₂₈F₃N₃O₃ (607.63)

Mass spectrum:(M−H)⁻606 (M+Na)⁺=630

### EXAMPLE 84

N-[4-(4-methyl-phenylcarbonylamino)-phenylmethyl]-3-(4′-trifluoromethylbiphenyl-2-carbonylamino)-benzoic acid amide

Prepared analogously to Example if from N-(4-aminophenylmethyl)-3-(4′-trifluoromethylbiphenyl-2-carbonylamino)-benzoic acid amide and 4-tolylic acid chloride in tetrahydrofuran with the addition of triethylamine.

Yield:95% of theory

R_{f} value:0.56 (silica gel; dichloromethane/ethanol=9:1)

C₃₆H₂₈F₃N₃O₃ (607.63)

Mass spectrum:(M−H)⁻=606 (M+Na)⁺=630

### EXAMPLE 85

N-(2′-dimethylaminocarbonylbiphenyl-4-methyl)-3-(4′-trifluoromethylbiphenyl-2-carbonylamino)-benzoic acid amide

Prepared analogously to Example 7 from N-(2′-hydroxycarbonylbiphenyl-4-methyl)-3-(4′-trifluoromethylbiphenyl-2-carbonylamino)-benzoic acid amide and dimethylamine in dichloromethane with the addition of propanephosphonic acid cycloanhydride and N-methylmorpholine.

Yield:24% of theory

R_{f} value:0.41 (silica gel; dichloromethane/ethanol=95:5)

C₃₇H₃₀F₃N₃O₃ (621.66)

Mass spectrum:(M−H)⁻620 (M+Na)⁺=644

### EXAMPLE 86

N-(2′-pyrrolidin-1-yl-carbonylbiphenyl-4-methyl)-3-(4′-trifluoromethylbiphenyl-2-carbonylamino)-benzoic acid amide

Prepared analogously to Example 7 from N-(2′-hydroxycarbonylbiphenyl-4-methyl)-3-(4′-trifluoromethylbiphenyl-2-carbonylamino)-benzoic acid amide and pyrrolidine in dichloromethane with the addition of propanephosphonic acid cycloanhydride and N-methylmorpholine.

Yield:92% of theory

R_{f} value:0.44 (silica gel; dichloromethane/ethanol=95:5)

C₃₉H₃₂F₃N₃O₃ (647.70)

Mass spectrum:(M−H)⁻=646 (M+Na)⁺=670

### EXAMPLE 87

N-[2′(2,2,2-trifluorethyl-aminocarbonyl)-biphenyl-4-methyl]-3-(4′-trifluoromethylbiphenyl-2-carbonylamino)-benzoic acid amide

Prepared analogously to Example 7 from N-(2′-hydroxycarbonylbiphenyl-4-methyl)-3-(4′-trifluoromethylbiphenyl-2-carbonylamino)-benzoic acid amide and 2,2,2-trifluoroethylamine in dichloromethane with the addition of propanephosphonic acid cycloanhydride and N-methylmorpholine.

Yield:18% of theory

R_{f} value:0.41 (silica gel; dichloromethane/ethanol=95:5)

C₃₇H₂₇F₆N₃O₃ (675.63)

Mass spectrum:(M−H)⁻=674 (M+Na)⁺=698

### EXAMPLE 88

N-[4-(Pyridin-2-yl-carbonylamino)-phenylmethyl]-3-(4′-trifluoromethylbiphenyl-2-carbonylamino)-benzoic acid amide

Prepared analogously to Example 1f from N-(4-aminophenylmethyl)-3-(4′-trifluoromethylbiphenyl-2-carbonylamino)-benzoic acid amide and pyridin-2-carboxylic acid chloride in tetrahydrofuran with the addition of triethylamine.

Yield:74% of theory

R_{f} value:0.64 (silica gel; dichloromethane/ethanol=9:1)

C₃₄H₂₅F₃N₄0₃ (594.59)

Mass spectrum:(M−H)⁻=593 (M+H)⁺=595 (M+Na)⁺=617

### EXAMPLE 89

N-(4′-methylbiphenyl-4-methyl)-2-methyl-3-(biphenyl-2-carbonylamino)-benzoic acid amide

Prepared analogously to Example if from N-(4′-methylbiphenyl-4-methyl)-3-amino-2-methyl-benzoic acid amide and biphenyl-2-carboxylic acid chloride in tetrahydrofliran with the addition of triethylamine.

Yield:22% of theory

R_{f} value:0.79 (silica gel; dichloromethane/ethanol=9:1)

C₃₅H₃₀N₂O₂ (510.64)

Mass spectrum:(M−H)⁻=509 (M+Na)⁺=533

### EXAMPLE 90

N-(biphenyl-4-methyl)-3-(biphenyl-2-carbonylamino)-5-nitro-benzoic acid amide

Prepared analogously to Example if from N-(biphenyl-4-methyl)-3-amino-5-nitro-benzoic acid amide and biphenyl-2-carboxylic acid chloride in tetrahydrofuran with the addition of triethylamine.

Yield:55% of theory

R_{f} value:0.88 (silica gel; dichloromethane/ethanol=9:1)

C₃₃H2₅N₃O₄ (527.58)

Mass spectrum:(M−H)⁻=526(M+Na)⁺=550

### EXAMPLE 91

N-(biphenyl-4-methyl)-3-(biphenyl-2-carbonylamino)-5-methyl-benzoic acid amide

Prepared analogously to Example if from N-(biphenyl-4-methyl)-3-amino-5-methyl-benzoic acid amide and biphenyl-2-carboxylic acid chloride in tetrahydrofuran with the addition of triethylamine.

Yield:5% of theory

R_{f} value:0.80 (silica gel; dichloromethane/ethanol=9:1)

C₃₄H₂₈N₂O₂ (496.61)

Mass spectrum:(M−H)⁻=495 (M+Na)⁺=519

### EXAMPLE 92

N-(biphenyl-4-methyl)-3-(biphenyl-2-carbonylamino)-4-fluor-benzoic acid amide

Prepared analogously to Example If from N-(biphenyl-4-methyl)-3-amino-4-fluorobenzoic acid amide and biphenyl-2-carboxylic acid chloride in tetrahydrofuran with the addition of triethylamine.

Yield:100% of theory

R_{f} value:0.45 (silica gel; dichloromethane)

C₃₃H₂₅FN₂O₂ (500.57)

Mass spectrum:(M−H)⁻=499 (M+Na)⁺=523

### EXAMPLE 93

N-(biphenyl-4-methyl)-5-amino-3-(biphenyl-2-carbonylamino)- benzoic acid amide 60 mg (0.11 mmol) of N-(biphenyl-4-methyl)-5-nitro-3-(biphenyl-2-carbonylamino)-benzoic acid amide are dissolved in 20 ml of methanol and 10 ml of dichloromethane and after the addition of 16 mg of palladium on activated charcoal (20%) hydrogenated with hydrogen for 3 hours at ambient temperature. The catalyst is filtered off and the solution is evaporated down.

Yield:56 mg (100% of theory),

R_{f} value:0.56 (silica gel; dichloromethane/ethanol=9:1)

C₃₃H₂₇N₃O₂ (497.60)

Mass spectrum:(M−H)⁻=496 (M+H)⁺=498 (M+Na)⁺=520

### EXAMPLE 94

N-(biphenyl-4-methyl)-5-(biphenyl-2-carbonylamino)-2-fluoro- benzoic acid amide

Prepared analogously to Example if from N-(biphenyl-4-methyl)-5-amino-2-fluorobenzoic acid amide and biphenyl-2-carboxylic acid chloride in tetrahydrofuran with the addition of triethylamine.

Yield:64% of theory

R_{f} value:0.68 (silica gel; dichloromethane/ethanol 9:1)

C₃₃H₂₅FN₂O₂ (500.57)

Mass spectrum:(M−H)⁻=499 (M+Na)⁺=523

### EXAMPLE 95

N-[4-(pyrazin-2-yl-carbonylamino)-phenylmethyl] -3-(4′-trifluoromethylbiphenyl-2-carbonylamino)-benzoic acid amide

Prepared analogously to Example if from N-(4-amino-phenylmethyl)-3-(4′-trifluoromethylbiphenyl-2-carbonylamino)-benzoic acid amide and pyrazin-2-carboxylic acid chloride in tetrahydrofuran with the addition of triethylamine.

Yield:22% of theory

R_{f} value:0.95 (silica gel; dichloromethane/ethanol=8:2)

C₃₃H₂₄F₃N₅0₃ (595.58)

Mass spectrum:(M−H)⁻=594 (M+Na)⁺=618

### EXAMPLE 96

N-[4-(pyrimidin-4-yl-carbonylamino)-phenylmethyl] -3-(4′-trifluoromethylbiphenyl-2-carbonylamino)-benzoic acid amide

Prepared analogously to Example 7 from N-(4-amino-phenylmethyl)-3-(4′-trifluoromethylbiphenyl-2-carbonylamino)-benzoic acid amide and pyrimidine-4-carboxylic acid in dichloromethane with the addition of propanephosphonic acid cycloanhydride and N-methylmorpholine.

Yield:29% of theory

R_{f} value:0.35 (silica gel; petroleum ether/ethyl acetate=3:7)

C₃₃H₂₄F₃N₅0₃ (595.58)

Mass spectrum:(M−H)⁻=594 (M+Na)⁺=618

### EXAMPLE 97

N-[4-(3-methyl-5-phenyl-pyrazol-1-yl)-phenylmethyl]-3-(6-methylbiphenyl-2-carbonylamino)-benzoic acid amide

Prepared analogously to Example 1f from N-[4-(3-methyl-5-phenyl-pyrazol-1-yl)-phenylmethyl]-3-amino-benzoic acid amide and 6-methylbiphenyl-2-carboxylic acid chloride in dimethylformamide with the addition of triethylamine.

Yield:24% of theory

R_{f} value:0.20 (silica gel; petroleum ether/ethyl acetate=1:1)

C₃₈H₃₂N₄O₂ (576.70)

Mass spectrum:(M−H)⁻=575 (M+H)⁺=577 (M+Na)⁺=599

### EXAMPLE 98

N-[3-(4-methylphenyl)-prop-2-ynyl]-3-(4′-trifluoromethyl-biphenyl-2-carbonylamino)-benzoic acid amide

Prepared analogously to Example 7 from 3-(4′-trifluoromethylbiphenyl-2-carbonylamino)-benzoic acid and 3-(4-methylphenyl)-prop-2-ynyl-amine in dichloromethane with the addition of propanephosphonic acid cycloanhydride and N-methylmorpholine.

Yield:24% of theory

R_{f} value:0.45 (silica gel; dichloromethane/ethanol=95:5)

C₃lH₂₃F₃N₂O₂ (512.53)

Mass spectrum:(M−H)⁻=511 (M+Na)⁺=535

### EXAMPLE 99

N-[3-(4-Isopropylphenyl)-prop-2-ynyl]-3-(4′-trifluoromethyl-biphenyl-2-carbonylamino)-benzoic acid amide

Prepared analogously to Example 7 from 3-(4′-trifluoromethylbiphenyl-2-carbonylamino)-benzoic acid and 3-(4-isopropylphenyl)-prop-2-ynyl-amine in dichloromethane with the addition of propanephosphonic acid cycloanhydride and N-methylmorpholine.

Yield:46% of theory

R_{f} value:0.43 (silica gel; dichloromethane/ethanol=95:5)

C₃₃H₂₇F₃N₂O₂ (540.59)

Mass spectrum:(M−H)⁻=539 (M+Na)⁺=563

### EXAMPLE 100

N-(biphenyl-4-methyl)-N-methyl-5-(biphenyl-2-carbonylamino)-2-methyl-benzoic acid amide

Prepared analogously to Example if from N-(biphenyl-4-methyl)-N-methyl-5-amino-2-methyl-benzoic acid amide and biphenyl-2-carboxylic acid chloride in tetrahydrofuran with the addition of triethylamine.

Yield:72% of theory

R_{f} value:0.50 (silica gel; dichloromethane/ethanol=19:1)

C₃₅H₃₀N₂O₂ (510.64)

Mass spectrum:(M−H)⁻=509 (M+Na)⁺=533

### EXAMPLE 101

N-(4-phenylamino-phenylmethyl)-3-(biphenyl-2-carbonylamino)-benzoic acid amide

Prepared analogously to Example 1f from 3-(biphenyl-2-carbonylamino)-benzoic acid and 4-phenylamino-benzylamine in tetrahydrofuran with the addition of triethylamine.

Yield:57% of theory

R_{f} value:0.50 (silica gel; dichloromethane/ethanol=9:1)

C₃₃H₂₇N₃O₂ (497.60)

Mass spectrum:(M+Na)⁺=520 M⁺=497

### EXAMPLE 102

N-(4-Morpholin-4-yl-phenylmethyl)-3-(biphenyl-2-carbonyl-amino)-benzoic acid amide

Prepared analogously to Example if from 3-(biphenyl-2-carbonylamino)-benzoic acid and 4-(morpholin-4-yl)-benzylamine in tetrahydrofuran with the addition of triethylamine.

Yield:27% of theory

R_{f} value:0.50 (silica gel; dichloromethane/ethanol=9:1)

C₃lH₂₉N₃O₃ (491.59)

Mass spectrum:(M+Na)⁺=514 (M−H)⁻=490 M⁺=491

### EXAMPLE 103

N-[4-(5-methylpyrazin-2-yl-carbonylamino]-phenylmethyl-3-(4′-trifluoromethylbiphenyl-2-carbonylamino)-benzoic acid amide

Prepared analogously to Example 7 from N-(4-amino-phenylmethyl)-3-(4′-trifluoromethylbiphenyl-2-carbonylamino)-benzoic acid amide and 5-methylpyrazine-2-carboxylic acid in dichloromethane with the addition of propanephosphonic acid cycloanhydride and N-methylmorpholine.

Yield:32% of theory

R_{f} value:0.14 (silica gel; petroleum ether/ethyl acetate=2:3)

C₃₄H₂₆F₃N₅O₃ (609.61)

Mass spectrum:(M−H)⁻608

### EXAMPLE 104

N-[4-(1H-pyrrol-2-yl-carbonylamino]-phenylmethyl-3-(4′-trifluoromethylbiphenyl-2-carbonylamino)-benzoic acid amide

Prepared analogously to Example 7 from N-(4-amino-phenylmethyl)-3-(4′trifluoromethylbiphenyl-2-carbonylamino)-benzoic acid amide and pyrrol-2-carboxylic acid in dichloromethane with the addition of propanephosphonic acid cycloanhydride and N-methylmorpholine.

Yield:14% of theory

R_{f} value:0.32 (silica gel; petroleum ether/ethyl acetate 2:3)

C₃₃H₂₅F₃N₄O₃ (582.58)

Mass spectrum:(M−H)⁻=581

### EXAMPLE 105

N-[3-(4-isopropylphenyl)-prop-2-ynyl]-3-(biphenyl-2-carbonylamino)-benzoic acid amide

Prepared analogously to Example 7 from 3-(biphenyl-2-carbonylamino)-benzoic acid and 3-(4-isopropylphenyl)-prop-2-ynyl-amine in dichloromethane with the addition of propanephosphonic acid cycloanhydride and N-methylmorpholine.

Yield:41% of theory

R_{f} value:0.42 (silica gel; dichloromethane/ethanol=95:5)

C₃₂H₂₈N₂O₂ (472.59)

Mass spectrum:(M−H)⁻=471 (M+Na)⁺=495

### EXAMPLE 106

N-[4-(N-methylpyrrol-2-yl)-carbonylaminophenylmethyl]-3-(4′-trifluoromethylbiphenyl-2-carbonylamino)-benzoic acid amide

Prepared analogously to Example if from N-(4-amino-phenylmethyl)-3-(4′-trifluoromethylbiphenyl-2-carbonylamino)-benzoic acid amide and N-methylpyrrol-2-carboxylic acid chloride in tetrahydrofuran with the addition of triethylamine.

Yield:58% of theory

R_{f} value:0.43 (silica gel; petroleum ether/ethyl acetate=2:3)

C₃₄H₂₇F₃N₄O₃ (596.61)

Mass spectrum:(M+Na)⁺=619 M⁺=596

### EXAMPLE 107

N-(4′-methylbiphenyl-4-methyl)-3-(4′-fluorobiphenyl-2-carbonylamino)-benzoic acid amide

Prepared analogously to Example 1f from N-(4′-methylbiphenyl-4-methyl)-3-amino-benzoic acid amide and 4′-fluorobiphenyl-2-carboxylic acid chloride in tetrahydrofuran with the addition of triethylamine.

Yield:74% of theory

R_{f} value:0.50 (silica gel; dichloromethane/ethanol=19:1)

C₃₄H₂₇FN₂O₂ (514.60)

Mass spectrum:(M−H)⁻=513 (M+Na)⁺=537

### EXAMPLE 108

N-(4′-trifluoromethylbiphenyl-4-methyl)-3-(biphenyl-2-carbonylamino)-benzoic acid amide

Prepared analogously to Example 1f from 3-(biphenyl-2-carbonylamino)-benzoic acid chloride and 4-(4-trifluoromethylphenyl)-benzylamine in tetrahydrofuran with the addition of triethylamine.

Yield:46% of theory

R_{f} value:0.65 (silica gel; dichloromethane/ethanol=9:1)

C₃₄H₂₅F₃N₂O₂ (550.58)

Mass spectrum:(M−H)⁻=549

### EXAMPLE 109

N-(4′-fluorobiphenyl-4-methyl)-3-(biphenyl-2-carbonylamino)-benzoic acid amide

Prepared analogously to Example 34 from 3-(biphenyl-2-carbonylamino)-benzoic acid, 4-(4-fluorophenyl)-benzylamine, TBTU and N-ethyldiisopropylamine in dimethylformamide.

Yield:89% of theory

R_{f} value:0.40 (silica gel; dichloromethane/ethanol=19:1)

C₃₃H2₅FN₂O₂ (500.57)

Mass spectrum:(M−H)⁻499 (M+Na)⁺=523

### EXAMPLE 110

N-[4-(pyridin-4-yl)-phenylmethyl]-3-(4′-trifluoromethyl-biphenyl-2-carbonylamino)-benzoic acid amide

Prepared analogously to Example 34 from 3-(4′-trifluoromethylbiphenyl-2-carbonylamino)-benzoic acid, 4-(pyridin-4-yl)-benzylamine, TBTU and N-ethyldiisopropylamine in dimethylformamide.

Yield:79% of theory

R_{f} value:0.5 (silica gel; dichloromethane/ethanol=9:1)

C₃₃H₂₄F₃N₃O₂ (551.57)

Mass spectrum:(M−H)⁻=550

### EXAMPLE 111

N-(4′-Chlorobiphenyl-4-methyl)-3-(4′-trifluoromethylbiphenyl-2-carbonylamino)-benzoic acid amide

Prepared analogously to Example 34 from 3-(4′-trifluoromethylbiphenyl-2-carbonylamino)-benzoic acid, 4-(4-chlorophenyl)-benzylamine, TBTU and N-ethyldiisopropylamine in dimethylformamide.

Yield:75% of theory

R_{f} value:0.0.6 (silica gel; dichloromethane/ethanol=9:1)

C₃₄H₂₄ClF₃N₂O₂ (585.03)

Mass spectrum:(M−H)⁻583/585 (chlorine isotopes)

### EXAMPLE 112

N-[3-(4-isopropylphenyl)propyl]-3-(4′-trifluoromethylbiphenyl-2-carbonylamino)-benzoic acid amide

Prepared analogously to Example 93 from N-[3-(4-isopropylphenyl)-prop-2-ynyl]-3-(4′trifluoromethylbiphenyl-2-carbonylamino)-benzoic acid amide and palladium on activated charcoal in ethanol.

Yield:99% of theory

R_{f} value:0.35 (silica gel; petroleum ether/ethyl acetate 3:2)

C₃₃H₃₁F₃N₂O₂ (544.62)

Mass spectrum:(M−H)⁻=543 (M+Na)⁺=567

### EXAMPLE 113

N— {4-[N-methyl-N-(3-methylphenyl)amino]-phenylmethyl} -3-(4′-trifluoromethylbiphenyl-2-carbonylamino)-benzoic acid amide

Prepared analogously to Example 1f from N— {4-[N-methyl-N-(3′-methylphenyl)amino]-phenylmethyl}-3-amino-benzoic acid amide and 4′-trifluoromethylbiphenyl-2-carboxylic acid chloride in tetrahydrofuran with the addition of triethylamine.

Yield:32% of theory

R_{f} value:0.60 (silica gel; dichloromethane/methanol=9:1)

C₃₆H₃₀F₃N₃O₂ (593.65)

Mass spectrum:(M−H)⁻=592 (M+Na)⁺=616

### EXAMPLE 114

N-[4-(1,2,3,4-tetrahydroquinolin-1-yl)-phenylmethyl]-3-(4′-fluoromethylbiphenyl-2-carbonylamino)-benzoic acid amide

Prepared analogously to Example if from N-[4-(1,2,3,4-tetrahydroquinolin-1-yl)-phenylmethyl]-3-amino-benzoic acid amide and 4′-fluoromethylbiphenyl-2-carboxylic acid chloride in tetrahydrofuran with the addition of triethylamine.

Yield:82% of theory

R_{f} value:0.80 (silica gel; dichloromethane/ethanol=9:1)

C₃₆H₃₀FN₃O₂ (555.65)

Mass spectrum:(M−H)⁻=554 (M+Na)⁺=578

### EXAMPLE 115

N-[4-(3,5-dimethyl-4-propyl-pyrazol-1-yl)-phenylmethyl]-3-(4′-trifluoromethylbiphenyl-2-carbonylamino)-benzoic acid amide

Prepared analogously to Example 1f from N-[4-(3,5-dimethyl-4-propyl-pyrazol-1-yl)-phenylmethyl]-3-amino-benzoic acid amide and 4′-trifluoromethylbiphenyl-2-carboxylic acid chloride in tetrahydrofuran with the addition of triethylamine.

Yield:66% of theory

R_{f} value:0.50 (silica gel; dichloromethane/ethanol=9:1)

C₃₆H₃₃F₃N₄0₂ (610.68)

Mass spectrum:(M−H)⁻=609 (M+H)⁺=611 (M+Na)⁺=633

### EXAMPLE 116

N-[4-(Imidazo-[1,2-a]pyridin-2-yl) phenylmethyl]-3-(4′-trifluoromethylbiphenyl-2-carbonylamino)-benzoic acid amide

Prepared analogously to Example 1f from N-[4-(imidazo-[1,2-a]pyridin-2-yl)phenylmethyl]-3-amino-benzoic acid amide and 4′-trifluoromethylbiphenyl-2-carboxylic acid chloride in tetrahydrofuran with the addition of triethylamine.

Yield:35% of theory

R_{f} value:0.65 (silica gel; dichloromethane/methanol=9:1)

C₃₅H₂₅F₃N₄O₂ (590.60)

Mass spectrum:(M−H)⁻589 (M+H)+=591 (M+Na)⁺=613

### EXAMPLE 117

N-[4-(1,2,3,4-tetrahydroquinolin-1-yl)-phenylmethyl] -3-(4′-trifluoromethylbiphenyl-2-carbonylamino)-benzoic acid amide

Prepared analogously to Example 1f from N-[4-(1,2,3,4-tetrahydro-quinolin-1-yl)-phenylmethyl]-3-amino-benzoic acid amide and 4′-trifluoromethylbiphenyl-2-carboxylic acid chloride in tetrahydrofuran with the addition of triethylamine.

Yield:60% of theory

R_{f} value:0.30 (silica gel; dichloromethane/ethanol=50:1)

C₃₇H₃₀F₃N₃O₂ (605.66)

Mass spectrum:(M−H)⁻=604 (M+Na)⁺=628

### EXAMPLE 118

N-[4-(1-methylbenzimidazol-2-yl)-phenylmethyl] -3-(4′-trifluoromethylbiphenyl-2-carbonylamino)-benzoic acid amide

Prepared analogously to Example 7 from 4-(1-methylbenzimidazol-2-yl)-benzylamine and 3-(4′-trifluoromethylbiphenyl-2-carbonylamino)-benzoic acid in dichloromethane with the addition of propanephosphonic acid cycloanhydride and N-methylmorpholine.

Yield:31% of theory

R_{f} value:0.70 (silica gel; dichloromethane/ethanol 19:1)

C₃₆H₂₇F₃N₄0₂ (604.63)

Mass spectrum:(M−H)⁻=603 (M+H)⁺=605 (M+Na)⁺=627

### EXAMPLE 119

N-(4-phenylcarbonylphenyl)-3-(4′-methylbiphenylcarbonylamino)-benzoic acid amide

Prepared analogously to Example If from N-(4-phenylcarbonylphenyl)-3-amino-benzoic acid amide and 4′-methylbiphenyl-2-carboxylic acid chloride in tetrahydrofuran with the addition of triethylamine.

Yield:90% of theory

R_{f} value:0.40 (silica gel; dichloromethane/ethanol 19:1)

C₃₄H₂₆N₂O₃ (510.59)

Mass spectrum:(M−H)⁻=509 (M+H)⁺=511 (M+Na)⁺=533

### EXAMPLE 120

N-(4-phenylaminocarbonylamino-phenylmethyl)-3-(4′-trifluoro-methylbiphenyl-2-carbonylamino)-benzoic acid amide 500 mg (0.95 mMol) of N-(4-amino-phenylmethyl)-3-(4′-trifluoro-methylbiphenyl-2-carbonylamino)-benzoic acid amide hydrochloride, 0.1 ml (1.05 mmol) of phenylisocyanate and 0.3 ml (2.3 mmol) of triethylamine are refluxed in 20 ml of tetrahydrofuran for 3 hours. The solvent is distilled off, the residue is dissolved in ethyl acetate and washed with 2 molar hydrochloric acid and 5% sodium hydrogen carbonate solution. The combined organic extracts are dried and evaporated down. The crude product is chromatographed on silica gel, eluting with dichloromethane/ethanol (1-3%).

Yield:97 mg (17% of theory),

R_{f} value:0.29 (silica gel; dichloromethane/ethanol=95:5)

C₃₅H₂₇F₃N₄0₃ (608.62)

Mass spectrum:(M−H)⁻=607 (M+Na)⁺=631

### EXAMPLE 121

N-(ethoxycarbonylmethyl-aminocarbonylmethyl)-3-(4′-trifluoromethylbiphenyl-2-carbonylamino)-benzoic acid amide

Prepared analogously to Example 34 from 3-(4′-trifluoromethyl-biphenyl-2-carbonylamino)-benzoic acid, glycylglycine ethylester, TBTU and N-ethyldiisopropylamine in dimethylformamide.

Yield:79% of theory

R_{f} value:0.49 (silica gel; petroleum ether/ethyl acetate=3:2)

C₂₇H₂₄F₃N₃O₅ (527.50)

Mass spectrum:(M−H)⁻=526 (M+H)⁺=528 (M+Na)⁺=550

### EXAMPLE 122

N-(biphenyl-4-methyl)-3-(9-oxofluorene-4-carbonylamino)-benzoic acid amide

Prepared analogously to Example 34 from 3-amino-N-(biphenyl-4-methyl)-benzoic acid amide, 9-oxofluorene-4-carboxylic acid, TBTU and N-ethyldiisopropylamine in dimethylformamide.

Yield:33% of theory

R_{f} value:0.37 (silica gel; dichloromethane/ethanol=95:5)

C₃₄H₂₄N₂O₃ (508.58)

Mass spectrum:(M−H)⁻=507 (M+Na)⁺=531

### EXAMPLE 123

N-(2-methyl-phenylmethyl)-3-(4′-trifluoromethylbiphenyl-2-carbonylamino)-benzoic acid amide

Prepared analogously to Example 34 from 3-(4′-trifluoromethyl-biphenyl-2-carbonylamino)-benzoic acid, 2-methylbenzylamine, TBTU and N-ethyldiisopropylamine in dimethylformamide.

Yield:82% of theory

R_{f} value:0.54 (silica gel; dichloromethane/ethanol=9:1)

C₂₉H₂₃F₃N₂O₂ (488.51)

Mass spectrum:(M−H)⁻487 (M+H)⁺=489 (M+Na)⁺=511

### EXAMPLE 124

N-[4-(6-methylpyridazin-3-yl)-phenylmethyl] -3-(4′-trifluoro-methylbiphenyl-2-carbonylamino)-benzoic acid amide

Prepared analogously to Example 34 from 3-(4′-trifluoromethyl-biphenyl-2-carbonylamino)-benzoic acid, 4-(6-methylpyridazin-3-yl)-benzylamine, TBTU and N-ethyldiisopropylamine in dimethylformamide.

Yield:36% of theory

R_{f} value:0.7 (silica gel; dichloromethane/ethanol=9:1)

C₃₃H₂₅F₃N₄O₂ (566.58)

Mass spectrum:(M−H)⁻=565 (M+H)⁺=567

### EXAMPLE 125

N-(2-Difluoromethoxy-phenylmethyl)-3-(4′-trifluoromethyl-biphenyl-2-carbonylamino)-benzoic acid amide

Prepared analogously to Example 34 from 3-(4′-trifluoromethyl-biphenyl-2-carbonylamino)-benzoic acid, 2-difluoromethoxybenzylamine, TBTU and N-ethyldiisopropylamine in dimethylformamide.

Yield:80% of theory

R_{f} value:0.69 (silica gel; dichloromethane/ethanol=19:1)

C₂₉H₂lF₅N₂O₃ (540.49) Mass spectrum:(M-H) 539 (M+H)⁺=541 (M+Na)⁺=563

### EXAMPLE 126

N—Cyclohexylmethyl-3-(4′-trifluoromethylbiphenyl-2-carbonyl-amino)-benzoic acid amide

Prepared analogously to Example 34 from 3-(4′-trifluoromethyl-biphenyl-2-carbonylamino)-benzoic acid, aminomethylcyclohexane, TBTU and N-ethyldiisopropylamine in dimethylformamide.

Yield:87% of theory

R_{f} value:0.72 (silica gel; dichloromethane/ethanol=9:1)

C₂₈H2₇F₃N₂O₂ (480.53)

Mass spectrum:(M−H)⁻=479 (M+H)⁺=481 (M+Na)⁺=503

### EXAMPLE 127

N-(9-fluorenyl)-3-(4′-trifluoromethylbiphenyl-2-carbonylamino)-benzoic acid amide

Prepared analogously to Example 34 from 3-(4′-trifluoromethyl-biphenyl-2-carbonylamino)-benzoic acid, 9-aminofluorene-hydrochloride, TBTU and N-ethyldiisopropylamine in dimethyl-formamide.

Yield:96% of theory

R_{f} value:0.75 (silica gel; dichloromethane/ethanol 19:1)

C₃₄H₂₃F₃N₂O₂ (548.57)

Mass spectrum:(M−H)⁻=547 (M+H)⁺=549 (M+Na)⁺=571

### EXAMPLE 128

N-(2-aminocarbonylethyl)-3-(4′-trifluoromethylbiphenyl-2-carbonyl-amino)-benzoic acid amide

Prepared analogously to Example 34 from 3-(4′-trifluoromethyl-biphenyl-2-earbonylamino)-benzoic acid, beta-alaninamide, TBTU and N-ethyldiisopropylamine in dimethylformamide.

Yield:61% of theory

R_{f} value:0.53 (silica gel; dichloromethane/ethanol=19:1)

C₂₄H₂₀F₃N₃O₃ (455.44)

Mass spectrum:(M−H)⁻=454 (M+H)⁺=456

### EXAMPLE 129

N-(1-aminocarbonyl-2-phenyl-ethyl)-3-(4′-trifluoromethyl-biphenyl-2-carbonylamino)-benzoic acid amide

Prepared analogously to Example 34 from 3-(4′-trifluoromethyl-biphenyl-2-carbonylamino)-benzoic acid, D,L-phenylalaninamide, TBTU and N-ethyldiisopropylamine in dimethylformamide.

Yield:73% of theory

R_{f} value:0.75 (silica gel; dichloromethane/ethanol=9:1)

C₃₀H₂₄F₃N₃O₃ (531.53)

Mass spectrum:(M−H)⁻=530 (M+H)⁺=532

### EXAMPLE 130

N-[4-(1,4-Dioxa-spiro[4.5]dec-8-yl)-phenyl]-3-(4′-trifluoromethylbiphenyl-2-carbonylamino)-benzoic acid amide

Prepared analogously to Example 34 from 3-(4′-trifluoromethyl-biphenyl-2-carbonylamino)-benzoic acid, 4-(1,4-dioxa-spiro[4.5]dec-8-yl)-phenylamine, TBTU and N-ethyldiisopropylamine in dimethylformamide.

Yield:90% of theory

R_{f} value:0.77 (silica gel; dichloromethane/ethanol=9:1)

C₃₅H₃₁F₃N₂O₄ (600.64)

Mass spectrum:(M−H)⁻=599 (M+H)⁺=601 (M+Na)⁺=623

### EXAMPLE 131

N-(1-phenylmethylaminocarbonyl-ethyl)-3-(4′-trifluoromethyl-biphenyl-2-carbonylamino)-benzoic acid amide

Prepared analogously to Example 34 from 3-(4′-trifluoromethyl-biphenyl-2-carbonylamino)-benzoic acid, 2-amino-N-benzyl-propionamide, TBTU and N-ethyldiisopropylamine in dimethylformamide.

Yield:73% of theory

R_{f} value:0.74 (silica gel; petroleum ether/ethyl acetate=3:2)

C₃₁H₂₆F₃N₃O₃ (545.56)

Mass spectrum:(M−H)⁻=544

### EXAMPLE 132

N-(aminocarbonylmethylaminocarbonylmethyl)-3-(4′-trifluoro-methylbiphenyl-2-carbonylamino)-benzoic acid amide

Prepared analogously to Example 34 from 3-(4′-trifluoromethyl-biphenyl-2-carbonylamino)-benzoic acid, H-Gly-Gly-NH₂ (glycylglycinamide), TBTU and N-ethyldiisopropylamine in dimethylformamide.

Yield:71% of theory

R_{f} value:0.75 (silica gel; petroleum ether/ethyl acetate=3:2)

C₂₅H₂₁F₃N₄0₄ (498.46)

Mass spectrum:(M−H)⁻=497 (M+H)⁺=499

### EXAMPLE 133

N-(1,2,3,4-tetrahydronaphthalin-1-yl)-3-(4′-trifluoromethyl-biphenyl-2-carbonylamino)-benzoic acid amide

Prepared analogously to Example 34 from 3-(4′-trifluoromethyl-biphenyl-2-carbonylamino)-benzoic acid, 1,2,3,4-tetrahydro-naphthalin-1-ylamine, TBTU and N-ethyldiisopropylamine in dimethylformamide.

Yield:72% of theory

R_{f} value:0.60 (silica gel; dichloromethane/ethanol=19:1)

C₃₁H₂₅F₃N₂O₂ (514.55)

Mass spectrum:(M−H)⁻=513 (M+H)⁺=515

### EXAMPLE 134

N-(4-tert.butoxycarbonylaminomethyl-phenylmethyl)-3-(4′-trifluoromethylbiphenyl-2-carbonylamino)-benzoic acid amide

Prepared analogously to Example 34 from 3-(4′-trifluoromethyl-biphenyl-2-carbonylamino)-benzoic acid, 4-(tert.butoxycarbonyl-aminomethyl)-benzylamine, TBTU and N-ethyldiisopropylamine in dimethylformamide.

Yield:89% of theory

R_{f} value:0.70 (silica gel; dichloromethane/ethanol 9:1)

C₃₄H₃₂F₃N₃O₄ (603.64)

Mass spectrum:(M−H)⁻=602 (M+Na)⁺=626

### EXAMPLE 135

N-(3-tert.-butoxycarbonylamino-propyl)-3-(4′-trifluoromethyl-biphenyl-2-carbonylamino)-benzoic acid amide

Prepared analogously to Example 34 from 3-(4′-trifluoromethyl-biphenyl-2-carbonylamino)-benzoic acid, 3-tert.-butoxycarbonyl-amino-propylamine, TBTU and N-ethyldiisopropyl-amine in dimethylformamide.

Yield:71% of theory

R_{f} value:0.50 (silica gel; dichloromethane/ethanol 19:1)

C₂₉H₃₀F₃N₃O₄ (541.57)

Mass spectrum:(M−H)⁻=540 (M+H)⁺=542

### EXAMPLE 136

N-(3-dimethylamino-propyl)-3-(4′-trifluoromethylbiphenyl-2-carbonylamino)-benzoic acid amide

Prepared analogously to Example 34 from 3-(4′-trifluoromethyl-biphenyl-2-carbonylamino)-benzoic acid, N,N-dimethyl- 1,3-propane-diamine, TBTU and N-ethyldiisopropylamine in dimethylformamide.

Yield:26% of theory

R_{f} value:0.08 (silica gel; dichloromethane/ethanol=9:1)

C₂₆H₂₆F₃N₃O₂ (469.51)

Mass spectrum:(M−H)⁻=468 (M+H)⁺=470

### EXAMPLE 137

N-[4-(1,2,3,4-tetrahydroisoquinolin-2-yl)-phenylmethyl]-3-(4′-trifluoromethylbiphenyl-2-carbonylamino)-benzoic acid amide

Prepared analogously to Example 34 from 3-(4′-trifluoromethyl-biphenyl-2-carbonylamino)-benzoic acid, 4-(1,2,3,4-tetrahydroisoquinolin-2-yl)-benzylamine, TBTU and N-ethyldiisopropylamine in dimethylformamide.

Yield:20% of theory

R_{f} value:0.30 (silica gel; dichloromethane/ethanol=50:1)

C₃₇H₃₀F₃N₃O₂ (605.66)

Mass spectrum:(M−H)⁻604 (M+HCOO)⁻650 (M+H)⁺=606

### EXAMPLE 138

N-[3-(6,7-dimethoxy-1,2,3,4-tetrahydroisoquinolin-2-yl-carbonyl)-phenyl]-4′-trifluoromethylbiphenyl-2-carboxylic acid amide

Prepared analogously to Example 34 from 3-(4′-trifluoromethyl-biphenyl-2-carbonylamino)-benzoic acid, 6,7-dimethoxy-1,2,3,4-tetrahydroisoquinoline, TBTU and N-ethyldiisopropylamine in dimethylformamide.

Yield:8% of theory

R_{f} value:0.50 (silica gel; dichloromethane/ethanol=19:1)

C₃₂H₂₇F₃N₂O₄ (560.57)

Mass spectrum:(M−H)⁻=559 (M+H)⁺=561 (M+Na)⁺=583

### EXAMPLE 139

N— {3-[5-dimethylaminomethylcarbonylamino-1,3-dihydro-isoindole-2-carbonyl]-phenyl} -4′-trifluoromethylbiphenyl-2-carboxylic acid amide

Prepared analogously to Example 34 from 3-(4′-trifluoromethyl-biphenyl-2-carbonylamino)-benzoic acid, N-(2,3-dihydro-1H-isoindol-5-yl)-2-dimethylamino-acetamide, TBTU and N-ethyldiisopropylamine in dimethylformamide.

Yield:87% of theory

R_{f} value:0.70 (silica gel; dichloromethane/ethanol 9:1)

C₃₃H₂₉F₃N₄0₃ (586.62)

Mass spectrum:(M−H)⁻585 (M+H)⁺=587

### EXAMPLE 140

N-[Cyclopropyl-(4-methoxy-phenyl)-methyl]-3-(4′-trifluoro-methyl-biphenyl-2-carbonylamino)-benzoic acid amide and N-[1-(4-methoxy-phenyl)-butyl]-3-(4′-trifluoromethylbiphenyl-2-carbonylamino)-benzoic acid amide in the ratio 1:1

Prepared analogously to Example 34 from 3-(4′-trifluoromethyl-biphenyl-2-carbonylamino)-benzoic acid, a 1:1 mixture of 1-(4-methoxy-phenyl)-n-butylamine and C-cyclopropyl-C-(4-methoxy-phenyl)-methylamine in the ratio 1:1, TBTU and N-ethyldiisopropylamine in dimethylformamide.

Yield:18% of theory

R_{f} value:0.80 (silica gel; petroleum ether/ethyl acetate=3:2)

N-[Cyclopropyl-(4-methoxy-phenyl)-methyl]-3-(4′-trifluoromethyl-biphenyl-2-carbonylamino)-benzoic acid amide

C₃₂H₂₇F₃N₂O₃ (544.58)

Mass spectrum:(M+H)⁺=545

N-[1-(4-methoxy-phenyl)-butyl] -3-(4′-trifluoromethylbiphenyl-2-carbonylamino)-benzoic acid amide

C₃₂H₂₉F₃N₂O₃ (546.59)

Mass spectrum:(M+H)⁺=547

### EXAMPLE 141

N-[1-(4-bromophenyl)ethyl]-3-(biphenyl-2-carbonylamino)-benzoic acid amide

Prepared analogously to Example 34 from 3-(biphenyl-2-carbonylamino)-benzoic acid, 1-(4-bromophenyl)ethylamine, TBTU and N-ethyldiisopropylamine in dimethylformamide. C₂₈H₂₃BrN₂O₂ (499.41)

Mass spectrum:(M−H)⁻=497/499 (bromine isotopes)

### EXAMPLE 142

N-[1-(4-Chloro-phenyl)ethyl]-3-(biphenyl-2-carbonylamino)-benzoic acid amide

Prepared analogously to Example 34 from 3-(biphenyl-2-carbonylamino)-benzoic acid, 1-(4-chlorophenyl)ethylamine, TBTU and N-ethyldiisopropylamine in dimethylformamide.

C₂₈H₂₃ClN₂O₂ (454.96)

Mass spectrum:(M−H)⁻=453

### EXAMPLE 143

N-[1-(2-naphtyl)ethyl]-3-(biphenyl-2-carbonylamino)-benzoic acid amide

Prepared analogously to Example 34 from 3-(biphenyl-2-carbonylamino)-benzoic acid, 1-(2-naphtyl)ethylamine, TBTU and N-ethyldiisopropylamine in dimethylformamide.

C₃₂H₂₆N₂O₂ (470.57)

Mass spectrum:(M+Na)⁺=469

### EXAMPLE 144

N-{2-[4-(4-hydroxyphenyl)phenyl]ethyl}-3-(biphenyl-2-carbonylamino)-benzoic acid amide

Prepared analogously to Example 34 from 3-(biphenyl-2-carbonylamino)-benzoic acid, 2-(4′-hydroxybiphenyl-4-yl)ethylamine, TBTU and N-ethyldiisopropylamine in dimethylformamide.

C₃₄H₂₈N₂O₃ (512.61)

Mass spectrum:(M−H)⁻511

### EXAMPLE 145

N-[2-(4-benzyloxyphenyl)ethyl]-3-(biphenyl-2-carbonylamino)-benzoic acid amide

Prepared analogously to Example 34 from 3-(biphenyl-2-carbonylamino)-benzoic acid, 2-(4-benzyloxyphenyl)ethylamine, TBTU and N-ethyldiisopropylamine in dimethylformamide.

C₃₅H₃₀N₂O₃ (526.63)

Mass spectrum:(M+H)⁺=527

### EXAMPLE 146

N-(Benzo[1,3]dioxol-5-ylmethyl)-3-(biphenyl-2-carbonylamino)-benzoic acid amide

Prepared analogously to Example 34 from 3-(biphenyl-2-carbonylamino)-benzoic acid, 3,4-(methylenedioxy)-benzylamine, TBTU and N-ethyldiisopropylamine in dimethylformamide.

C₂₈H₂₂N₂O₄ (450.49)

Mass spectrum:(M−H)⁻=449

The following compounds may be prepared analogously and by methods known from the literature:

(1) N-(3,4-dimethoxy-phenylmethyl)-3-(4′-trifluoromethylbiphenyl-2-carbonylamino)-benzoic acid amide

(2) N-[3-(4-biphenylyl)-prop-2-enyl]-3-(4′-trifluoromethylbiphenyl-2-carbonylamino)-benzoic acid amide

(3) N-(4-aminomethyl-phenylmethyl)-3-(4′-trifluoromethylbiphenyl-2-carbonylamino)-benzoic acid amide

(4) N-[4-(1,4-dioxa-spiro[4,5]dec-8-yl)-phenylmethyl]-3-(4′-trifluoromethylbiphenyl-2-carbonylamino)-benzoic acid amide

(5) N-[4-(1 ,4-dioxa-8-aza-spiro[4,5]dec-8-yl)-phenylmethyl]-3-(4′-trifluoromethylbiphenyl-2-carbonylamino)-benzoic acid amide

(6) N-[4-(4-oxocyclohexyl)-phenylmethyl]-3-(4′-trifluoromethylbiphenyl-2-carbonylamino)-benzoic acid amide

(7) N-[4-(5-dimethylaminopyridin-2-yl)-phenylmethyl]-3-(4′-trifluoromethylbiphenyl-2-carbonylamino)-benzoic acid amide

(8) N-(2-dimethylaminoethyl)-3-(4′-trifluoromethylbiphenyl-2-carbonylamino)-benzoic acid amide

(9) N-[3-(4-biphenylyl)-prop-2-ynyl]-3-(4′-trifluoromethylbiphenyl-2-carbonylamino)-benzoic acid amide

(10) N-[3-(4-tert.butylphenyl)-prop-2-ynyl]-3-(4′-trifluoromethylbiphenyl-2-carbonylamino)-benzoic acid amide

(11) N-[3-(4-tert.butylphenyl)-propyl]-3-(4′-trifluoromethylbiphenyl-2-carbonylamino)-benzoic acid amide

(12) N-[3-(4-biphenylyl)-propyl] -3-(4′-trifluoromethylbiphenyl-2-carbonylamino)-benzoic acid amide

### EXAMPLE 147

Tablets containing 5 mg of active substance per tablet

Composition:

| | |
|---|---|
| active substance | 5.0 mg |
| lactose monohydrate | 70.8 mg |
| microcrystalline cellulose | 40.0 mg |
| sodium carboxymethylcellulose, insolubly crosslinked | 3.0 mg |
| magnesium stearate | 1.2 mg |

Preparation:

The active substance is mixed for 15 minutes with lactose monohydrate, microcrystalline cellulose and sodium carboxymethylcellulose in a suitable diffusion mixer. Magnesium stearate is added and mixed with the other substances for another 3 minutes. The finished mixture is compressed in a tablet press to form facetted flat round tablets.

Diameter of the tablet:7 mm

Weight of the tablet:20 mg

### EXAMPLE 148

Capsules containing 50 mg of active substance per capsule

Composition:

| | |
|---|---|
| active substance | 50.0 mg |
| lactose monohydrate | 130.0 mg |
| corn starch | 65.0 mg |
| highly dispersed silicon dioxide | 2.5 mg |
| magnesium stearate | 2.5 mg |

Preparation:

A starch paste is prepared by allowing some of the corn starch to swell in a suitable amount of hot water. The paste is then left to cool to room temperature. The active substance is premixed for 15 minutes in a suitable mixer with lactose monohydrate and corn starch. The starch paste is added and the mixture is mixed with sufficient water to produce a moist homogeneous mass. The moist mass is passed through a screen with a mesh size of 1.6 mm. The screened granules are dried on racks at about 55° C. for 12 hours.

The dried granules are then passed through screens with mesh sizes of 1.2 and 0.8 mm. Highly dispersed silica is mixed with the granules in a suitable mixer for 3 minutes. Then magnesium stearate is added and mixing is continued for another 3 minutes.

The finished mixture is packed into empty size 1 hard gelatine capsule shells using a capsule filling machine.

### EXAMPLE 149

Tablets containing 200 mg of active substance per tablet

Composition:

| | |
|---|---|
| active substance | 200.0 mg |
| lactose-monohydrate | 167.0 mg |
| microcrystalline cellulose | 80.0 mg |
| hydroxypropyl-methylcellulose, type 2910 | 10.0 mg |
| poly-1-vinyl-2-pyrrolidone, insolubly crosslinked | 20.0 mg |
| magnesium stearate | 3.0 mg |

Preparation:

HPMC is dispersed in hot water. After cooling, the mixture yields a clear solution.

The active substance is premixed in a suitable mixer for 5 minutes with lactose monohydrate and microcrystalline cellulose. The HPMC solution is added and the mixing is continued until a homogeneous moist composition is obtained. The moist composition is passed through a screen with a mesh size of 1.6 mm. The screened granules are dried on racks at about 55° C. for 12 hours.

The dried granules are then passed through screens with mesh sizes of 1.2 and 0.8 mm. Poly-1-vinyl-2-pyrrolidone is mixed with the granules in a suitable mixer for 3 minutes.

Then magnesium stearate is added and mixing is continued for another 3 minutes.

The finished mixture is compressed in a tablet press to form oblong tablets (16.2×7.9 mm).

Weight of a tablet:480 mg

We claim:

1. A Biphenylcarboxylic acid amide of the following formula (I): [chemical structure]
wherein
R¹, R2 and R³, which may be identical or different, in each case denote a hydrogen, fluorine, chlorine or bromine atom, a straight-chain or branched C₁₋₃-alkyl group wherein the hydrogen atoms may be wholly or partially replaced by fluorine atoms, a hydroxy, C₁₋₃-alkoxy, amino, C₁₋₃-alkylamino- or di-(C₁₋₃-alkyl)-amino group,
wherein R¹ and R² in the ortho,ortho′ position of the biphenyl group of formula I together may also denote a carbonyl group,
R⁴ denotes a hydrogen atom or a C₁₋₃-alkyl group,
R⁵ denotes a hydrogen atom or a straight-chain or branched C₁₋₆-alkyl group and
R⁶ denotes a straight-chain or branched C₁₋₆-alkyl group,
an amino, C₁₋₃-alkylamino or di-(C₁₋₃-alkyl)-amino group,
a C₃₋₇-cycloalkylamino or N-(C₁₋₃-alkyl)-C₃₋₇-cycloalkyl-amino group, wherein
in each case the methylene group in the 4 position of a 6- or 7-membered cycloalkyl group may be replaced by an oxygen or sulphur atom or by an imino group optionally substituted by a C₁₋₃-alkyl, phenyl, C₁₋₃-alkyl-carbonyl, benzoyl, phenyl-(C₁₋₃-alkyl)-carbonyl, C₁₋₃-alkyl-aminocarbonyl, di-(C₁₋₃-alkyl)-aminocarbonyl, phenylaminocarbonyl or N-(C₁₋₃-alkyl)-phenylaminocarbonyl group,
an arylamino, N-(C₁₋₃-alkyl)-arylamino, heteroarylamino, N-(C₁₋₃-alkyl)-heteroarylamino, C₁₋₇-alkyl-carbonylamino, N-(C₁-3-alkyl)-C₁₋₇-alkyl-carbonylamino, arylcarbonylamino, heteroarylcarbonylamino, N-(C₁₋₃-alkyl)-arylcarbonylamino, N-(C₁₋₃-alkyl)-heteroarylcarbonylamino, C₁₋₈-alkoxy-carbonyl-amino or N-(C₁₋₃-alkyl)-(C₁₋₈-alkoxy)-carbonylamino group,
an aryl, aryl-carbonyl-aryl, aryl-C₁₋₃-alkoxy-aryl or aryl-C₁₋₃-alkyl-aryl group,
a heteroaryl group,
an aryl group substituted by a heteroaryl group,
a C₃₋₇-cycloalkyl or C₃₋₇-cycloalkyl-aryl group, while
in each case the methylene group in the 4 position of a 6- or 7-membered cycloalkyl group may be replaced by an oxygen or sulphur atom or by an imino group optionally substituted by a C₁₋₃-alkyl, phenyl, C₁₋₃-alkyl-carbonyl, benzoyl, phenyl-(C₁₋₃-alkyl)-carbonyl, C₁₋₃-alkyl-aminocarbonyl, di-(C₁₋₃-alkyl)-aminocarbonyl, phenylaminocarbonyl or N-(C₁₋₃-alkyl)-phenylaminocarbonyl group, or
the two hydrogen atoms of the methylene group in the 3 position of a cyclopentyl group or in the 3- or 4-position of a cyclohexyl or cycloheptyl group may be replaced by an n-butylene, n-pentylene, n-hexylene, 1,2-ethylenedioxy or 1,3-propylenedioxy group or
in a 5- or 6-membered cycloalkyl group one or two single bonds separated from each other and from position 1 by at least one bond may each be fused with a phenyl group,
a phenylcarbonylamino-aryl, phenylaminocarbonyl-aryl, N-(C₁₋₃-alkyl)-phenylcarbonylamino-aryl or N-(C₁₋₃-alkyl)-phenylaminocarbonyl-aryl group,
a straight-chain C₁₋₄-alkyl group optionally substituted in the 1 position by a C₃₋₅-cycloalkyl group or a C₁₋₃-alkyl group, which is terminally substituted
by an aryl or heteroaryl group,
by an aryl—C C—, heteroaryl—C C—, aryl—CH═CH— or heteroaryl-CH═CH— group,
by an aryl group which is fused to a heteroaryl group via two adjacent carbon atoms,
by a heteroaryl group which is fused to an aryl or heteroaryl group via two adjacent carbon atoms or, in the case of a 5-membered heteroaryl group, via an imino nitrogen atom and an adjacent carbon atom,
by an aryl group which is substituted
by an aryl or heteroaryl group,
by a C₃₋₇-cycloalkyl group or a 4 to 7 membered cycloalkyleneimino group, which
may each be fused to a phenyl ring via two adjacent carbon atoms or
wherein the two hydrogen atoms of the methylene group in the 3 position of a 5-membered ring or in position 3 or 4 of a 6- or 7-membered ring may be replaced by an n-butylene,
n-pentylene, n-hexylene, 1,2-ethylenedioxy or 1,3-propylenedioxy group or by an oxygen atom or
wherein in each case the methylene group in the 4 position of a 6- or 7-membered ring may be replaced by an oxygen or sulphur atom or by an imino group optionally substituted by a C₁₋₃-alkyl, phenyl, C₁₋₈-alkyl-carbonyl, C₁₋₈-alkoxycarbonyl, benzoyl, phenyl-(C₁₋₃-alkyl-carbonyl), C₁₋₃-alkyl-aminocarbonyl, di-(C₁₋₃-alkyl)-aminocarbonyl, phenylaminocarbonyl or N-(C₁₋₃-alkyl)-phenylaminocarbonyl group,
or by a phenylaminosulphonyl or phenylsulphonylamino group,
by a C₃₋₇-cycloalkyl group wherein
in each case the methylene group in the 4 position of a 6- or 7-membered cycloalkyl group may be replaced by an oxygen or sulphur atom or by an imino group optionally substituted by a C₁₋₃-alkyl, phenyl, C₁₋₈-alkyl-carbonyl, C₁₋₈-alkoxycarbonyl, benzoyl, phenyl-(C₁₋₃-alkylcarbonyl), C₁₋₃-alkylaminocarbonyl, di-(C₁₋₃-alkyl)-aminocarbonyl, phenylaminocarbonyl or N-(C₁₋₃-alkyl)-phenylaminocarbonyl group,
by a phenylcarbonylamino-aryl, phenylaminocarbonyl-aryl, N-(C₁₋₃-alkyl)-phenylcarbonylamino-aryl or N-(C₁₋₃-alkyl)-phenylaminocarbonyl-aryl group,
by a heteroarylcarbonylamino-aryl, heteroarylaminocarbonyl-aryl, heteroarylcarbonyl-N-(C₁₋₃-alkyl)-amino-aryl or heteroaryl-N-(C₁₋₃-alkyl)-aminocarbonyl-aryl group, by a straight-chain or branched C₄₋₇-alkyl-carbonylamino-aryl or N-(C₁₋₃-alkyl)-C₄₋₇-alkyl-carbonylamino-aryl group,
by a C₃₋₇-cycloalkyl-carbonylamino-aryl or N-(C₁₋₃-alkyl)-C_{3 7}-cycloalkyl-carbonylamino-aryl group,
by a C₃₋₇-Cycloalkyl-aminocarbonyl-aryl or N-(C₁₋₃-alkyl)-C₃₋₇-cycloalkyl-aminocarbonyl-aryl group,
by a cycloalkyleneimino-carbonylamino-aryl or cycloalkyleneimino-carbonyl-N-(C₁₋₃-alkyl)-amino-aryl group wherein the cycloalkyleneimino moiety is 4- to 7-membered,
by an aryl-aminocarbonylamino-aryl group wherein one or both amino-hydrogen atoms may each be replaced by a C₁₋₃-alkyl group,
by a hydroxycarbonyl, C₁₋₃-alkoxycarbonyl, C₃₋₇-cyclo-alkyloxycarbonyl, aryloxycarbonyl, heteroaryloxycarbonyl, aryl-C₁₋₃-alkoxycarbonyl or heteroaryl-C₁₋₃-alkoxycarbonyl group or
by an aminocarbonyl, C₁₋₃-alkyl-aminocarbonyl, aryl-C₁₋₃-alkyl-aminocarbonyl, N-(C₁₋₃-alkyl)-aryl-C₁₋₃-alkyl-aminocarbonyl, di-(C₁₋₃-alkyl)-aminocarbonyl, aminocarbonyl-C₁₋₃-alkyl-aminocarbonyl or C₁₋₃-alkoxy-carbonyl-C₁₋₃-alkyl-aminocarbonyl group,
a straight-chain or branched C₂₋₆-alkyl group which is terminally substituted
by a hydroxy, C₁₋₃-alkoxy, aryloxy, heteroaryloxy- aryl-C₁₋₃-alkoxy or heteroaryl-C₁₋₃-alkoxy group,
by an amino, C₁₋₃-alkylamino, di-(C₁₋₃-alkyl)-amino, C₁₋₃-alkyl-carbonylamino, N-(C₁₋₃-alkyl)-C₁₋₃-alkylcarbonylamino, arylcarbonylamino, heteroarylcarbonylamino, N-(C₁₋₃-alkyl)-arylcarbonylamino or N-(C₁₋₃-alkyl)-heteroarylcarbonylamino group,
or R⁵ and R⁶ together with the enclosed nitrogen atom denote a 4- to 7-membered cycloalkyleneimino group wherein the cycloalkylene moiety may be fused to a phenyl ring,
R⁷ denotes a hydrogen, fluorine, chlorine, bromine or iodine atom, a C₁₋₃-alkyl,
C₁₋₃-alkoxy, nitro or amino group,
wherein by the term aryl group mentioned above is meant a phenyl, 1-naphthyl or 2-naphthyl group,
by the term heteroaryl group mentioned above is meant a 5-membered heteroaromatic ring linked via a nitrogen or carbon atom, which contains
an imino group, an oxygen or sulphur atom,
an imino group and an oxygen, sulphur or nitrogen atom,
an imino group and two nitrogen atoms or
an oxygen or sulphur atom and two nitrogen atoms,
or a 6-membered heteroaromatic ring linked via a carbon atom which contains one or two nitrogen atoms,
and wherein a 1,4-butadienylene group may be attached both to the abovementioned 5-membered heteroaromatic rings via two adjacent carbon atoms or via an imino nitrogen atom and an adjacent carbon atom and also to the 6-membered heteroaromatic rings in each case via two adjacent carbon atoms and the bicyclic heteroaromatic rings thus formed may also be bonded via a carbon atom of the 1,4- butadienylene group,
a hydrogen atom bonded to a nitrogen atom of the abovementioned 5-membered monocyclic or fused heteroaryl groups may be replaced by a C₁₋₃-alkyl, phenyl, phenyl-C₁₋₃-alkyl, C₁₋₃-alkylcarbonyl, phenylcarbonyl or phenyl-C₁₋₃-alkylcarbonyl group,
all the abovementioned phenyl, aryl and heteroaryl groups as well as aromatic or heteroaromatic parts of molecules in the carbon skeleton may be monosubstituted by a fluorine, chlorine or bromine atom, by a straight-chain or branched C₁₋₄-alkyl group, by a C₃₋₇-cycloalkyl or a 4- to 7-membered cycloalkyleneimino group, while
in each case the methylene group in position 4 of a 6- or 7-membered cycloalkyleneimino group may be replaced by an oxygen or sulphur atom, by a sulphinyl or sulphonyl group or by an imino group optionally substituted by a C₁₋₅-alkyl, phenyl, C₁₋₄-alkyl-carbonyl, C₁₋₄-alkoxy-carbonyl, C₁₋₃-alkyl-aminocarbonyl or di-(C₁₋₃-alkyl)-aminocarbonyl group,
by a trifluoromethyl, phenyl, hydroxy, C₁₋₃-alkoxy, phenyl-C₁₋₃-alkoxy, difluoromethoxy, trifluoromethoxy, amino, C₁₋₃-alkylamino, di-(C₁₋₃-alkyl)-amino, amino-C₁₋₃-alkyl, tert.butoxycarbonylamino-C₁₋₃-alkyl, C₁₋₃-alkylamino-C₁₋₃-alkyl, di-(C₁₋₃-alkyl)-amino-C₁₋₃-alkyl, amino-C₁₋₃-alkyl-carbonyl-amino, C₁₋₃-alkylamino-C₁₋₃-alkyl-carbonyl-amino, di-(C₁₋₃-alkyl)-amino-C₁₋₃-alkyl-carbonyl-amino, phenylamino, N-(C₁₋₃-alkyl)-phenylamino, acetylamino, propionylamino, benzoylamino, N-(C₁₋₃-alkyl)-benzoylamino, acetyl, propionyl, benzoyl, hydroxycarbonyl, C₁₋₄-alkoxycarbonyl, aminocarbonyl, C₁₋₃-alkylamino-carbonyl, 2,2,2-trifluoroethyl-ami-nocarbonyl or di-(C₁₋₃-alkyl)aminocarbonyl group, by a 4- to 7-membered cycloalkyleneimino-carbonyl group or a cyano group or, with the exception of 5-membered heteroaryl groups or heteroaromatic parts of molecules containing more than two heteroatoms, may also be disubstituted by one of the abovementioned substituents and one substituent selected from among fluorine, chlorine, bromine, C₁₋₃-alkyl, trifluoromethyl, C₁₋₃-alkoxy, hydroxy and amino, wherein two adjacent hydrogen atoms in a phenyl group or a phenyl moiety contained in the groups defined above may also be replaced by a methylenedioxy or 1,2-ethylenedioxy group, or may also be trisubstituted by three substituents selected from among fluorine, chlorine and bromine atoms and C₁₋₃-alkyl groups, wherein the substituents may be identical or different and the abovementioned phenyl groups or phenyl moieties may in turn be substituted by a fluorine, chlorine or bromine atom, by a methyl, trifluoromethyl or methoxy group,
in all the abovementioned 4- to 7-membered cycloalkyleneimino groups the cycloalkylene moiety may be fused to a phenyl ring or
one or two hydrogen atoms in each case may be replaced by a C₁₋₃-alkyl group and/or
in each case the methylene group in position 4 of a 6- or 7-membered cycloalkyleneimino group may be substituted by a hydroxycarbonyl, C₁₋₆-alkoxycarbonyl, aminocarbonyl, C₁₋₃-alkylamino-carbonyl, di-(Cl ₃-alkyl)-aminocarbonyl, phenyl-C₁₋₃-alkylamino or N-(C₁₋₃-alkyl)-phenyl-C₁₋₃-alkylamino group or
may be replaced by an oxygen or sulphur atom, by a sulphinyl or sulphonyl group or by an imino group optionally substituted by a C₁₋₃-alkyl, phenyl, C₁₋₃-alkyl-carbonyl, benzoyl, phenyl-C₁₋₃-alkyl-carbonyl, Cl ₃-alkyl-aminocarbonyl, di-(C₁₋₃-alkyl)-aminocarbonyl, phenylaminocarbonyl or N-(C₁₋₃-alkyl)-phenylaminocarbonyl group,
the hydrogen atoms in the C₁₋₃-alkyl and alkoxy groups mentioned in the definition of the above groups may be wholly or partially replaced by fluorine atoms,
additionally any carboxy, amino or imino group present in the abovementioned groups may be substituted by a group which can be cleaved in vivo,
or a tautomer, a diastereomer, or an enantiomer, or mixtures thereof or a salt thereof.

2. A compound of formula I according to claim 1, wherein
R¹ denotes a hydrogen, fluorine, chlorine or bromine atom or a C₁₋₃-alkyl group wherein the hydrogen atoms may be wholly or partially replaced by fluorine atoms,
R² denotes a hydrogen atom or a C₁₋₃-alkyl group or
R₁ and R² in the ortho, ortho′ position of the biphenyl group of formula I together denote a carbonyl group,
R³, R⁴ and R⁵ which may be identical or different, each denote a hydrogen atom or a C₁₋₃-alkyl group,
R⁶ denotes a straight-chain or branched C₁₋₄-alkyl group,
an amino, C₁₋₃-alkylamino or di-(C₁₋₃-alkyl)-amino group,
a C₃₋₇-cycloalkylamino or N-(C₁₋₃-alkyl)-C₃₋₇-cycloalkyl-amino group, wherein
in each case the methylene group in the 4 position of the cyclohexyl group may be replaced by an oxygen or sulphur atom or by an imino group optionally substituted by a C₁₋₃-alkyl, phenyl, C₁₋₃-alkyl-carbonyl, C₁₋₈-alkoxy-carbonyl, benzoyl, C₁₋₃-alkyl-aminocarbonyl, di-(C₁₋₃-alkyl)-aminocarbonyl, phenyl-aminocarbonyl or N-(C₁₋₃-alkyl)-phenylaminocarbonyl group,
a phenylamino, 1-naphthylamino or 2-naphthylamino group optionally substituted at the nitrogen atom by a C₁₋₃-alkyl group,
a C₁₋₄-alkyl-carbonylamino, phenylcarbonylamino or C₁₋₈-alkoxy-carbonylamino group,
a phenyl, biphenyl, 1-naphthyl, 2-naphthyl, phenylcarbonyl-phenyl, phenyl-C₁₋₃-alkoxyphenyl or phenyl-C₁₋₃-alkylphenyl group which may be substituted in the aromatic moieties in each case by a fluorine, chlorine, bromine or iodine atom, by a straight-chain or branched C₁₋₄-alkyl group, by a trifluoromethyl, hydroxy, C₁₋₃-alkoxy,
amino, C₁₋₃-alkylamino, di-(C₁₋₃-alkyl)-amino, acetylamino, benzoylamino, acetyl, benzoyl, C₁₋₃-alkylamino-carbonyl or cyano group,
a heteroaryl group or a heteroaryl-phenyl group,
a C₃₋₇-cycloalkyl or C₃₋₇-cycloalkyl-phenyl group, wherein
in each case the methylene group in the 4 position of the cyclohexyl group may be replaced by an oxygen or sulphur atom or by an imino group optionally substituted by a C₁₋₃-alkyl, phenyl, C₁₋₃-alkylcarbonyl, benzoyl, C₁₋₃-alkyl-aminocarbonyl, di-(C₁₋₃-alkyl)-aminocarbonyl, phenylaminocarbonyl or N-(C₁₋₃-alkyl)-phenylaminocarbonyl group, or
the two hydrogen atoms of the methylene group in the 3 position of a cyclopentyl group or in the 4-position of a cyclohexyl group may be replaced by an n-butylene, n-pentylene, 1,2-ethylenedioxy or 1,3-propylenedioxy group or
in a cyclopentyl or cyclohexyl group one or two single bonds separated from each other and from position 1 by at least one bond may each be fused to a phenyl group,
a phenylcarbonylamino-phenyl, phenylaminocarbonyl-phenyl, N-(C₁₋₃-alkyl)-phenylcarbonylamino-phenyl or N-(C₁₋₃-alkyl)-phenylaminocarbonyl-phenyl group,
a straight-chain C₁₋₄-alkyl group optionally substituted in the 1 position by a cyclopropyl group or a C₁₋₃-alkyl group, which is terminally substituted
by a phenyl, biphenyl, 1-naphthyl or 2-naphthyl group optionally substituted by a fluorine, chlorine, bromine or iodine atom, a straight-chain or branched C₁₋₄-alkyl group, a trifluoromethyl, hydroxy, C₁₋₃-alkoxy, difluoromethoxy, benzyloxy, aminomethyl, amino, C₁₋₃-alkylamino, di-(C₁₋₃-alkyl)-amino, phenylamino, N-(C₁₋₃-alkyl)-phenylamino,
acetylamino, acetyl, propionyl, benzoyl, hydroxycarbonyl, C₁₋₄-alkoxycarbonyl, aminocarbonyl, C₁₋₃-alkylamino-carbonyl, di-(C₁₋₃-alkyl)aminocarbonyl, 2,2,2-trifluoroethylaminocarbonyl, pyrrolidinocarbonyl, piperidinocarbonyl or cyano group wherein two adjacent hydrogen atoms may also be replaced by a methylenedioxy or 1,2-ethylenedioxy group,
by a heteroaryl group optionally substituted in the carbon skeleton by a fluorine, chlorine, bromine or iodine atom, by a straight-chain or branched C₁₄-alkyl or C₁₋₃-alkoxy group, by a trifluoromethyl, phenyl or cyano group,
by a phenyl—C C— or phenyl—CH═CH— group which may be substituted in the phenyl moiety by a fluorine, chlorine, bromine or iodine atom, by a straight-chain or branched C₁₋₄-alkyl or C₁₋₃-alkoxy group, by a trifluoromethyl, dimethylamino, phenyl or cyano group,
by an indolyl, benzimidazolyl, quinolinyl, isoquinolinyl, quinoxalinyl or quinazolinyl group bonded via a carbon atom or, in the case of the first two groups, via a nitrogen atom,
by a phenyl group which is substituted by a heteroaryl group optionally substituted in the carbon skeleton by a fluorine, chlorine, bromine or iodine atom, by a straight-chain or branched C₁₋₄-alkyl group, by a C₃₋₇-cycloalkyl, trifluoromethyl, phenyl or cyano group,
by a C₅₋₆-cycloalkyl group or a 5- or 6-membered cycloalkyleneimino group which
may be fused to a phenyl ring in each case via two adjacent carbon atoms or
wherein the two hydrogen atoms of the methylene group in the 3 position of a 5-membered ring or in the 4 position of a 6-membered ring may be replaced by an n-butylene, n-pentylene, n-hexylene, 1,2-ethylenedioxy or 1,3-propylenedioxy group or by an oxygen atom or
wherein the methylene group in the 4 position of a 6-membered ring may be replaced by an oxygen or sulphur atom or by an imino group optionally substituted by a C₁₋₃-alkyl, phenyl, C₁₋₄-alkyl-carbonyl, C₁₋₄-alkoxy-carbonyl or benzoyl group,
by a phenylaminosulphonylphenyl or phenylsulphonyl-aminophenyl group,
by a C3-7-cycloalkyl group, wherein
in each case the methylene group in the 4 position of the cyclohexyl group may be replaced by an oxygen or sulphur atom or by an imino group optionally substituted by a C₁₋₃-alkyl, phenyl, C₁₋₃-alkyl-carbonyl, benzoyl, C₁₋₃-alkyl-aminocarbonyl, di-(C₁₋₃-alkyl)-aminocarbonyl, phenylaminocarbonyl or N-(C₁₋₃-alkyl)-phenylaminocarbonyl group,
by a phenylcarbonylamino-phenyl, phenylaminocarbonyl-phenyl,
N-(C₁₋₃-alkyl)-phenylcarbonylamino-phenyl or
N-(C₁₋₃-alkyl)-phenylaminocarbonyl-phenyl group, phenyl-C₁₋₃-alkyl-aminocarbonyl-phenyl,
N-(C₁₋₃-alkyl)-phenyl-C₁₋₃-alkyl-aminocarbonyl-phenyl,
C₃₋₇-Cycloalkyl-carbonylamino-phenyl,
N-(C₁₋₃-alkyl)-C₃₋₇-aycloalkyl-carbonylamino-phenyl,
C₃₋₇-cycloalkyl-aminocarbonyl-phenyl,
N-(C₁₋₃-alkyl)-C₃₋₇-cycloalkyl-aminocarbonyl-phenyl, C₄₋₆-alkyl-carbonylamino-phenyl,
N-(C₁₋₃-alkyl)-C₄₋₆-alkyl-carbonylamino-phenyl, heteroarylcarbonylamino-phenyl,
N-(C₁₋₃-alkyl)-heteroarylearbonylamino-phenyl, pyrrolidinocarbonyl-amino-phenyl,
piperidinocarbonyl-amino-phenyl, N-(C₁₋₃-alkyl)-pyrrolidinocarbonyl-amino-phenyl,
N-(C₁₋₃-alkyl)-piperidinocarbonyl-amino-phenyl, phenylaminocarbonylamino-phenyl,
N-(C₁₋₃-alkyl)-phenylaminocarbonylamino-phenyl or
N,N-di-(C₁₋₃-alkyl)-phenylaminocarbonylamino-phenyl group,
by a hydroxycarbonyl, C₁₋₃-alkoxycarbonyl, phenyloxycarbonyl or heteroaryloxycarbonyl group,
by an aminocarbonyl, C₁₋₃-alkyl-aminocarbonyl, benzyl-aminocarbonyl, di-(C₁₋₃-alkyl)-aminocarbonyl, aminocarbonyl-C₁₋₃-alkyl-aminocarbonyl or C₁₋₃-alkoxy-carbonyl-C₁₋₃-alkyl-aminocarbonyl group,
a straight-chain C₂₋₃-alkyl group which is terminally substituted
by a hydroxy, C₁₋₃-alkoxy, phenoxy or phenyl-C₁₋₃-alkoxy group or
by an amino, C₁₋₃-alkylamino, di-(C₁₋₃-alkyl)-amino, C₁₋₃-alkyl-carbonylamino, N-(C₁₋₃-alkyl)-C₁₋₃-alkyl-carbonylamino, phenylcarbonylamino or N-(C₁₋₃-alkyl)phenylcarbonylamino group,
or R⁵ and R⁶ together with the enclosed nitrogen atom denote a pyrrolidino or piperidino group which
may each be fused via two adjacent carbon atoms to a phenyl ring optionally substituted by one or two C₁₋₃-alkoxy groups, by an amino, C₁₋₃-alkylamino, acetylamino, aminomethylcarbonylamino or dimethylaminomethylcarbonylamino group,
or a piperazino, morpholino or thiomorpholino group, while the nitrogen atom in the 4 position of the piperazino group may be substituted by a C₁₋₃-alkyl, phenyl, C₁₋₃-alkylcarbonyl, benzoyl, C₁₋₃-alkyl-aminocarbonyl or phenylaminocarbonyl group, and
R⁷ denotes a hydrogen, fluorine, chlorine or bromine atom or a C₁₋₃-alkyl group or a nitro or amino group,
while, unless otherwise specified, by the term heteroaryl group mentioned above is meant a 2-pyridyl, 3-pyridyl, 4-pyridyl, pyrazinyl, 2-pyrimidinyl, 4-pyrimidinyl, 5-pyrimidinyl, 3-pyridazinyl, 4-pyridazinyl, 1-pyrrolyl, 2-pyrrolyl, 3-pyrrolyl, 2-furyl, 3-furyl, 2-thienyl, 3-thienyl, 1-imidazolyl, 2-imidazolyl, 4-imidazolyl, 1-pyrazolyl, 3-pyrazolyl, 4-pyrazolyl, 2-thiazolyl, 4-thiazolyl, 5-thiazolyl, [1,2,3]-thiadiazol-4-yl, benzimidazol-2-yl, benzimidazol-5-yl, or imidazo-[1,2-a]pyridin-2-yl group optionally substituted in the carbon skeleton by up to three C₁₋₃-alkyl groups and
all the abovementioned phenyl groups, heteroaryl groups, aromatic or heteroaromatic parts of molecules may optionally additionally be substituted in the carbon skeleton by a fluorine, chlorine or bromine atom, by a cyano group or by a straight-chain or branched C₁₋₃-alkyl or trifluoromethyl group,
and/or a hydrogen atom bonded to a nitrogen atom of a heteroaryl group or heteroaromatic part of a molecule may be replaced by a C₁₋₃-alkyl, phenyl or C₁₋₃-alkylcarbonyl group,
or a tautomer, a diastereomer, or an enantiomer, or mixtures thereof or a salt thereof.

3. A compound of formula I according to claim 1, wherein
R¹ denotes a hydrogen, fluorine, chlorine or bromine atom, a C₁₋₃-alkyl or trifluoromethyl group,
R² denotes a hydrogen atom or a C₁₋₃-alkyl group or
R¹ and R² in the ortho, ortho′ position of the biphenyl group of formula I may together also denote a carbonyl group,
R³ and R⁴ each denote a hydrogen atom,
R⁵ denotes a hydrogen atom or a C₁₋₃-alkyl group,
R⁶ denotes a straight-chain or branched C₁₋₄-alkyl group,
a phenyl, biphenyl or phenyl-C₁₋₃-alkylphenyl group,
a straight-chain C₁₋₃-alkyl group optionally substituted in the 1 position by a cyclopropyl group or a C₁₋₃-alkyl group which is terminally substituted
by a phenyl or biphenyl group which may be substituted in each case by a fluorine, chlorine or bromine atom, by a straight-chain or branched C₁₋₄-alkyl group, by a trifluoromethyl, hydroxy, phenylamino or N-(C₁₋₃-alkyl)-phenylamino group,
by a 2-pyridyl, 3-pyridyl, 4-pyridyl or 1H-benzimidazol-2-yl group,
by a phenyl group which is substituted by a 2-pyridyl, 3-pyridyl, 4-pyridyl, pyrazinyl, 2-pyrimidinyl, 4-pyrimidinyl, 5-pyrimidinyl, 3-pyridazinyl, 4-pyridazinyl, 1-pyrrolyl, 2-pyrrolyl, 3-pyrrolyl, 1-imidazolyl, 2-imidazolyl, 4-imidazolyl, 1-pyrazolyl, 3-pyrazolyl, 4-pyrazolyl, 2-thiazolyl, 4-thiazolyl, 5-thiazolyl, [1,2,3]-thiadiazol-4-yl, benzimidazol-2-yl or imidazo-[1,2-a]pyridin-2-yl group, wherein the abovementioned heteroaromatic groups may be substituted in the carbon skeleton by a fluorine, chlorine or bromine atom, by a phenyl, C₁₋₄-alkyl, trifluoromethyl, C₁₋₃-alkoxy, dimethylamino or C₃₋₇-cycloalkyl group,
by a phenyl group which is substituted by a pyrrolidino or piperidino group optionally fused to a phenyl group,
by a phenyl—C C— group which may be substituted in the phenyl moiety by a fluorine, chlorine or bromine atom, by a straight-chain or branched C₁₋₄-alkyl or C₁₋₃-alkoxy group, by a trifluoromethyl or phenyl group,
by a 4-piperidinyl group optionally substituted at the nitrogen atom by a C₁₋₃-alkyl, C₁₋₃-alkyl-carbonyl, benzoyl, C₁₋₃-alkylaminocarbonyl, di-(C₁₋₃-alkyl)-aminocarbonyl, phenylamino-carbonyl or N-(C₁₋₃-alkyl)-phenylaminocarbonyl group,
by a phenylcarbonylamino-phenyl, phenylaminocarbonyl-phenyl,
N-(C₁₋₃-alkyl)-phenylcarbonylamino-phenyl or
N-(C₁₋₃-alkyl)-phenylaminocarbonyl-phenyl group optionally substituted in the terminal phenyl moieties by a C₁₋₃-alkyl group
or
by a heteroaryl-carbonylamino-phenyl or N-(C₁₋₃-alkyl)-heteroaryl-carbonylamino-phenyl group, wherein the heteroaryl moiety is selected from among 2-pyridyl, 3-pyridyl, 4-pyridyl, pyrazinyl, 2-pyrimidinyl, 4-pyrimidinyl, 5-pyrimidinyl, 3-pyridazinyl, 4-pyridazinyl, 1-pyrrolyl, 2-pyrrolyl, 3-pyrrolyl, 1-imidazolyl, 2-imidazolyl, 4-imidazolyl, 1-pyrazolyl, 3-pyrazolyl, 4-pyrazolyl, 2-thiazolyl, 4-thiazolyl, 5-thiazolyl and [1,2,3]-thiadiazol-4-yl, wherein a hydrogen atom bound to a nitrogen atom of a heteroaromatic group and/or a hydrogen atom bound to a carbon atom of a heteroaromatic group may in each case be replaced by a C₁₋₃-alkyl group, and
R⁷ denotes a hydrogen, fluorine, chlorine or bromine atom, a C₁₋₃-alkyl group or an amino group,
while all the abovementioned phenyl groups, heteroaryl groups, aromatic or heteroaromatic parts of molecules in the carbon skeleton may optionally additionally be substituted by a fluorine, chlorine or bromine atom, by a straight-chain or branched C₁₋₃-alkyl group, by a cyano or a trifluoromethyl group,
or a tautomer, a diastereomer, or an enantiomer, or mixtures thereof or a salt thereof.

4. A compound of formula I according to claim 1 selected from the group consisting of:
(a) N-[4-(3-Methyl-5-phenyl-pyrazol-1-yl)-phenylmethyl]-3-(4′-trifluoromethylbiphenyl-2-carbonylamino)-benzoic acid amide;
(b) N-(4′-Methylbiphenyl-4-methyl)-3-(biphenyl-2-carbonylamino)-benzoic acid amide,
(c) N-[4-(Pyridin-2-yl-carbonylamino)-phenylmethyl]-3-(4′-trifluoromethylbiphenyl-2-carbonylamino)-benzoic acid amide,
(d) N-[3-(4-Isopropylphenyl)-prop-2-ynyl]-3-(4′-trifluoromethylbiphenyl-2-carbonylamino)-benzoic acid amide and
(e) N-[4-(1,2,3 ,4-Tetrahydroquinolin-1-yl)-phenylmethyl]-3-(4′-trifluoromethylbiphenyl-2-carbonylamino)-benzoic acid amide,
and the salts thereof.

5. A compound according to any one of claims 1 to 4 in the form of its physiologically acceptable salt.

6. A pharmaceutical composition comprising a compound according to any one of claims 1 to 4, or a physiologically acceptable salt thereof, together with one or more inert carriers and/or diluents.

7. A method of lowering the plasma level of atherogenic lipoproteins in a subject in need thereof, comprising adminstering to said subject an effective amount of a compound of formula I according to any one of claims 1 to 4 or a physiologically acceptable salt thereof.

8. A method of treating hyperlipidaemias, atherosclerosis or the clinical sequelae thereof, diabetes mellitus, adiposity or pancreatitis in a subject in need thereof, comprising adminstering to said subject an effective amount of a compound of formula I according to any one of claims 1 to 4 or a physiologically acceptable salt thereof.
